(19) United States
(12) Patent Application Publication
(10) Pub. No.: US 2009/0075935 A1
(43) Pub. Date: Mar. 19, 2009

(54) COMPOSITION COMPRISING AT LEAST ONE C-GLYCOSIDE DERIVATIVE AND AT LEAST ONE HYALURONIC ACID AND ITS COSMETIC USE

(75) Inventors: Laure BISSEY, Paris (FR); Julien LABOUREAU, Paris (FR); Jerome SENEE, Lardy (FR); Sophie SAUSSAY, Survilliers (FR)

Correspondence Address: OLIFF & BERRIDGE, PLC, P.O. BOX 320850, ALEXANDRIA, VA, 22320-4850, US

(73) Assignee: L'OREAL, Paris (FR)

(21) Appl. No.: 12/192,698

(22) Filed: Aug. 15, 2008

Related U.S. Application Data
(63) Continuation of application No. PCT/FR2007/051579, filed on Jul. 2, 2007.
(60) Provisional application No. 60/968,892, filed on Aug. 30, 2007.
(60) Provisional application No. 60/817,683, filed on Jul. 3, 2006.
(60) Provisional application No. 60/894,254, filed on Mar. 12, 2007.

(51) Int. Cl. A61K 8/73; A61Q 19/08

(52) U.S. Cl. 514/54

(57) ABSTRACT

The present invention relates to a topical cosmetic and/or dermatological composition comprising, in a physiologically acceptable medium, at least one hyaluronic acid or a derivative thereof and at least one C-glycoside derivative.

The present invention relates to the field of skincare and/or the care of skin appendages.

A subject of the invention is a topical cosmetic and/or dermatological composition comprising, in a physiologically acceptable medium, at least one hyaluronic acid or a derivative thereof and at least one C-glycoside derivative.

The present invention also relates to a cosmetic treatment process for the skin and/or appendages thereof, intended to improve the barrier function, comprising at least one step consisting in applying at least one composition as defined above to the skin.

In particular, the composition of the invention is for improving the barrier function of the skin. It can thus be used in hydrating the skin, in improving the suppleness of the skin, in improving and/or decreasing the microrelief of the skin, and also in combating the signs of aging of the skin.

Human skin consists of two compartments, namely a deep compartment, the dermis, and a superficial compartment, the epidermis.

The dermis provides the epidermis with a solid support. It is also the feeder element of the epidermis. It consists mainly of fibroblasts and of an extracellular matrix which is itself composed mainly of collagen, elastin and a substance known as ground substance, these components being synthesized by the fibroblasts. Leukocytes, mast cells or tissue macrophages are also found in the dermis. It also contains blood vessels and nerve fibers.

The epidermis is in contact with the outside environment.

Natural human epidermis is composed mainly of three types of cells, which are the keratinocytes, which form the vast majority, the melanocytes and the Langerhans cells.

The cells constituting the epidermis are delimited by an intercellular lipid domain.

Each of these cell types contributes, by virtue of its own functions, to the essential role played by the skin in the organism. In particular, the keratinocytes undergo a process of continuous and oriented maturation which, from the keratinocytes that are in the basal layer of the epidermis, results in the formation of corneocytes, which are completely keratinized dead cells consisting of keratinocytes in the terminal stage of their differentiation.

During differentiation, the phospholipids, the role of which consists in producing the fluid structure of the cell membranes of the living layers of the epidermis, are gradually replaced with a mixture composed pre-dominantly of fatty acids, cholesterol and sphingolipids (ceramides). These lipids, which are organized in specific lamellar liquid crystal phases, form the intracellular cement of the stratum corneum and are essential for the water exchanges and the barrier function of the epidermis. Thus, the lamellar structure of the lipids of the lipid domain of the epidermis and the corneocytes participate in the epidermal barrier function.

The skin thus constitutes a barrier against outside attacks, in particular chemical, mechanical or infectious attacks, and, in this respect, a certain number of defense reactions against environmental factors (climate, ultraviolet rays, tobacco, etc.) and/or xenobiotics, for instance microorganisms, occur therein.

This property, called barrier function, is mainly provided by the most superficial layer of the epidermis, namely the horny layer, called the stratum corneum.

It is clear that the quality and the equilibrium of the skin barrier and the mucous membranes is dependent on complex endogenous biological mechanisms involving many growth factors, adhesion molecules, hormones and lipid metabolism enzymes.

Thus, an impairment of the skin barrier can occur in the presence of outside attacks such as irritants (detergents, acids, bases, oxidants, reducing agents, concentrated solvents, toxic gases or fumes), mechanical stresses (friction, impacts, abrasion, tearing of the surface, projection of dust or of particles, shaving or hair removal), heat or climatic imbalances (cold, dryness, radiation), xenobiotics (undesirable microorganisms, allergens) or internal attacks such as psychological stress.

Individuals to whom this impairment of the barrier function by outside attacks more particularly relates may be the following:
individuals with “fragile” or “delicate” and vulnerable skin which rapidly becomes unbalanced during large variations in temperature or in relative humidity (in the case of the skin of babies, for example);
individuals with “weakened” skin, which groups together in particular
individuals in whom the protective aqueous-lipid film composed of sweat, sebum and natural moisturizing factors becomes scarce, as is the case in individuals over the age of 60, and in particular in the context of old age (at least 75 years old);
individuals in whom the composition of the aqueous-lipid film is modified, as is the case of diabetic individuals, individuals who undergo dialysis, or individuals suffering from certain diseases;
individuals who have a reduced reactivity threshold due to neurogenic hyperactivity; this skin will therefore exhibit these sensations and these clinical signs much more rapidly and frequently than other types of skin: these are individuals with sensitive skin.

Reference may also be made to individuals with “stressed” skin for skin that has been shaved, for example.

An impairment of the barrier function of the skin may in particular be reflected by a problem of hydration, by a loss of suppleness of the skin, by an impaired radiance of the complexion, by the appearance of roughness on the skin, and more generally by the appearance of signs of skin aging.

It is therefore advisable to seek to increase the epidermal differentiation in order to reinforce the barrier function of the skin.

In particular, it is thus sought to improve and/or reinforce the barrier function of the skin in order to:
overcome the problems of hydration of the skin and/or of its appendages, in particular the mucous membranes, and in particular treat dry skin,
improve the suppleness of the skin,
maintain and/or improve the radiance of the complexion,
prevent and/or treat the roughness or microrelief of the skin which can manifest itself through chickenpox or acne marks,
fade out the cutaneous signs and/or the cutaneous imperfections associated with aging of the skin, among which mention may be made of impairment of the viscoelastic or biomechanical properties of the skin, impairment of tissue cohesion, thinning of the skin, the appearance of wrinkles and/or fine lines, the appearance of browning and/or yellowing of the skin, and the appearance of age or senescence or lentigo spots.

The signs of aging of the skin may be of actinic origin.

Sugars and sugar derivatives are products that have already been used to advantage for various purposes for the formulation of cosmetic compositions intended both for skincare and for care and/or washing of keratin fibers.

Thus, in document WO 99/24009, D-xylose and derivatives thereof are proposed for the purposes of preparing cosmetic or pharmaceutical products aimed at improving the functionality of epidermal cells.

Among the sugars that can be used in the field, C-glycoside derivatives prove to be most particularly advantageous. These C-glycoside derivatives have especially demonstrated advantageous biological properties, in particular for combating aging of the epidermis and/or combating dryness of the skin. Such compounds are in particular described in document WO 02/051828.

These compounds act via the stimulation of the synthesis of glycosaminoglycans containing a D-glucosamine and/or N-acetyl-D-glucosamine residue and are represented by the formula:

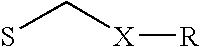

in which S represents a monosaccharide or a polysaccharide, R represents various linear or cyclic radicals and the group X may represent a group chosen from: —CO—, —CH(NR₁R₂)—, —CHR₁— or —C(═CHR₃)— with it being possible for R₁, R₂, R′ and R₃ to represent various radicals, including the hydroxyl radical for R₁, R₂ and R₃.

However, these derivatives had up until now never been used for the purposes of improving and/or reinforcing the barrier function of the skin, and even less in combination with hyaluronic acid.

Hyaluronic acid is a predominant glucosaminoglycan found in the skin. Thus, the fibroblasts synthesize predominantly collagens, matrix glycoproteins other than collagens (fibronectin, laminin), proteoglycans and elastin. The keratinocytes, for their part, synthesize predominantly sulfated glycosaminoglycans and hyaluronic acid. Hyaluronic acid is also called hyaluronan (HA).

Hyaluronic acid is present in the free state in the epidermis and in the dermis and is responsible for turgescence of the skin. This polysaccharide can in fact retain a large volume of water, corresponding to up to 1000 times its weight. In this sense, hyaluronic acid plays an important role in increasing the amounts of water bound in the tissue, and also in the mechanical properties of the skin and in wrinkle formation.

The inventors have discovered that certain C-glycoside derivatives combined with hyaluronic acid or a derivative thereof make it possible to improve and/or reinforce the barrier function of the skin.

The inventors have in particular demonstrated that the combination in accordance with the invention has a synergistic effect on the activity of keratinocyte transglutaminase (TGk). This combination thus makes it possible to improve epidermal homeostasis.

The combination in accordance with the present invention has the property of activating TGk, an essential factor in terminal differentiation of keratinocytes and of cornification thereof.

TGk is an enzyme belonging to the transpeptidase family. It is a calcium-dependent enzyme which catalyzes the formation of ε(γ-glutamyl) lysine isopeptide bridges between epidermal proteins and therefore promotes the formation of cornified envelopes.

According to a first of its aspects, the invention thus relates to a topical cosmetic and/or dermatological composition comprising, in a physiologically acceptable medium, at least one hyaluronic acid or a derivative thereof and at least one C-glycoside derivative.

According to another of its aspects, it also relates to the cosmetic use of a composition according to the invention or else of at least one C-glycoside derivative and of at least one hyaluronic acid or a derivative thereof in a topical composition for improving and/or reinforcing the cutaneous barrier function of the skin.

According to another of its aspects, it also relates to the cosmetic use of a composition according to the invention of else of at least one C-glycoside derivative and of at least one hyaluronic acid or a derivative thereof in a topical composition, for improving and/or reinforcing the protection of the skin against outside attacks.

According to yet another of its aspects, it also relates to the cosmetic use of a composition according to the invention or else of at least one C-glycoside derivative and of at least one hyaluronic acid or a derivative thereof in topical compositions, for improving the hydration of the skin and/or its appendages, preventing and/or treating the roughness or the microrelief and/or for improving the radiance of the complexion and/or for improving the suppleness of the skin and/or its appendages and/or for fading out the signs of skin aging.

The invention also relates to a cosmetic treatment process for improving and/or reinforcing the barrier function and/or the hydration and/or the resistance to outside attacks of the skin and/or its appendages, and/or for combating the signs of skin aging, and characterized in that an effective amount of at least one C-glycoside derivative and of at least one hyaluronic acid or a derivative thereof or else a composition according to the invention is applied to the skin and/or its appendages.

More particularly, the process is aimed at reducing the signs of skin aging of actinic origin.

It is in particular intended for individuals with mature or even very mature skin.

The term “mature skin” according to the invention is intended to mean in particular individuals at least 40 years old.

The term “very mature skin” according to the invention is intended to mean in particular individuals at least 50 years old, in particular at least 60 years old, or even 65 years old.

According to another embodiment, said composition may be for improving and/or reinforcing the barrier function of skin chosen from fragile skin, weakened skin, stressed skin and/or sensitive skin.

In the context of the invention, the composition may be used for application to normal skin, which is subjected or which may be subjected to outside attacks as recalled above. In other specific cases, the composition of the invention may be applied to the skin when it shows clinical signs of cutaneous barrier deficiency.

A subject of the invention is thus the use of at least one hyaluronic acid or a derivative thereof and of at least one C-glycoside derivative, for the preparation of a topical, in particular dermatological, composition for improving and/or reinforcing the barrier function of the skin.

Another aspect of the present invention is therefore the use of at least one hyaluronic acid or a derivative thereof and of at least one C-glycoside derivative, for the preparation of a topical, in particular dermatological, composition for improving and/or reinforcing the barrier function of damaged skin, in particular of skin requiring tissue repair and/or regeneration.

Finally, a subject of the invention is the combination of at least one hyaluronic acid or of at least one derivative thereof and of at least one C-glycoside derivative, for its use in improving and/or reinforcing the barrier function of damaged skin, in particular skin requiring tissue repair and/or regeneration.

The composition according to the invention may in particular be for improving healing or stimulating tissue repair and/or regeneration.

Tissue repair may prove to be necessary in all cases where the skin shows lesions of any origin, in particular due to damage caused by UV radiation or outside attacks of pollution type or a stress.

In the context of the present invention, the expression “viscoelastic or biomechanical properties of the skin” is intended to mean the properties of extendability, tonicity, firmness, suppleness and/or elasticity of the skin. Skin showing such an impairment of its viscoelastic or biomechanical properties may be described as soft skin, flaccid skin, less firm skin, less elastic skin, or else skin that has sagged.

The impairment of the “tissue cohesion” manifests itself through the impairment of the biomechanical properties of the skin, thinning of the skin and a decrease in the cell metabolism of the skin.

The term “skin and/or its appendages” is intended to mean in particular the skin, the mucous membranes, the lips, the scalp, the eyelashes, the eyebrows and the hair.

The term “effective amount” is intended to mean respective amounts of two active agents which allow the manifestation of the desired synergistic effect.

The combination according to the invention may be formulated in the same topical composition or in two separate topical compositions, which may be applied to the skin and/or its appendages simultaneously, successively or sequentially over time.

### Hyaluronic Acid

In the context of the present invention, the term “hyaluronic acid or a derivative thereof/” covers in particular the basic unit of hyaluronic acid of formula:

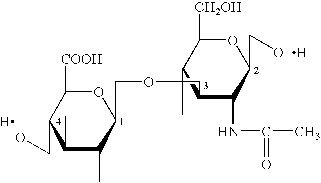

It is the smallest fraction of hyaluronic acid comprising a disaccharide dimer, namely D-glucuronic acid and N-acetylglucosamine.

The term “hyaluronic acid or a derivative thereof” also comprises, in the context of the present invention, the linear polymer comprising the polymeric unit described above, linked together in the chain via alternating β(1,4) and β(1,3) glycosidic linkages, having a molecular weight (MW) that can range between 380 and 13 000 000 daltons. This molecular weight depends in large part on the source from which the hyaluronic acid is obtained and/or on the preparation methods.

The term “hyaluronic acid or a derivative thereof” also comprises, in the context of the present invention, the hyaluronic acid salts, and in particular the alkali metals salts such as the sodium salt and the potassium salt.

In the natural state, hyaluronic acid is present in pericellular gels, in the base substance of the connective tissues of vertebrate organs such as the dermis and epithelial tissues, and in particular in the epidermis, in the synovial fluid of the joints, in the vitreous humor, in the human umbilical cord and in the crista galli apophysis.

Thus, the term “hyaluronic acid or a derivative thereof” comprises all the fractions or subunits of hyaluronic acid having a molecular weight in particular within the molecular weight range recalled above.

In the context of the present invention, hyaluronic acid fractions which do not have an inflammatory activity are preferably used.

By way of illustration of the various hyaluronic acid fractions, reference may be made to the document “Hyaluronan fragments: an information-rich system”, R. Stern et al., European Journal of Cell Biology 58 (2006) 699-715, which reviews the listed biological activities of hyaluronic acid according to its molecular weight.

According to a preferred embodiment of the invention the hyaluronic acid fractions suitable for the use covered by the present invention have a molecular weight of between 50 000 and 5 000 000, in particular between 100 000 and 5 000 000, especially between 400 000 and 5 000 000 Da. In this case, the term used is high-molecular-weight hyaluronic acid.

Alternatively, the hyaluronic acid fractions that may also be suitable for the use covered by the present invention have a molecular weight of between 50 000 and 400 000 Da. In this case, the term used is intermediate-molecular-weight hyaluronic acid.

Alternatively again, the hyaluronic acid fractions that may be suitable for the use covered by the present invention have a molecular weight of less than 50 000 Da. In this case, the term used is low-molecular-weight hyaluronic acid.

Finally, the term “hyaluronic acid or a derivative thereof” also comprises hyaluronic acid esters in particular those in which all or some of the carboxylic groups of the acid functions are esterified with oxyethylenated alkyls or alcohols, containing from 1 to 20 carbon atoms, in particular with a degree of substitution at the level of the D-glucuronic acid of the hyaluronic acid ranging from 0.5 to 50%.

Mention may in particular be made of methyl, ethyl, n-propyl, n-pentyl, benzyl and dodecyl esters of hyaluronic acid. Such esters have in particular been described in D. Campoccia et al. “Semisynthetic resorbable materials from hyaluronan esterification”, Biomaterials 19 (1998) 2101-2127.

The molecular weights indicated above are also valid for the hyaluronic acid esters.

Hyaluronic acid may in particular be hyaluronic acid supplied by the company Hyactive under the trade name CPN (MW: 10 to 150 kDa), by the company Soliance under the trade name Cristalhyal (MW: 1.1×10⁶), by the company Bioland under the name Nutra HA (MW: 820 000 Da), by the company Bioland under the name Nutra AF (MW: 69 000 Da, by the company Bioland under the name Oligo HA (MW: 6100 Da) or else by the company Vam Farmacos Metica under the name D Factor (MW: 380 Da).

In one embodiment, the hyaluronic acid is present in the form of spheres. In particular, such spheres are sold by the company BASF under the name Sphere d'Acide Hyaluronique [hyaluronic acid sphere]. It is a mixture of hyaluronic acid of various molecular weights, i.e. of MW 1.5×10⁶, 400 000 and 600 000 Da.

The hyaluronic acid or a derivative thereof is present in the composition according to the present invention at a content of between 0.001% and 20%, preferably between 0.01% and 10%, and more particularly between 0.01% and 5% by weight, relative to the total weight of the composition.

### C-Glycoside Derivatives

A C-glycoside derivative suitable for the invention may be a compound of general formula (I) below:

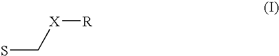

in which:
R represents:
   a C₁ to C₂₀, in particular C₁ to C₁₀, saturated or C₂ to C₂₀, in particular C₂ to C₁₀, unsaturated, linear alkyl radical, or a C₃ to C₂₀, in particular C₃ to C₁₀, saturated or unsaturated, branched or cyclic alkyl radical;
   a C₁ to C₂₀, in particular C₁ to C₁₀, saturated or C₂ to C₂₀, in particular C₂ to C₁₀, unsaturated, linear, or C₃ to C₂₀, in particular C₃ to C₁₀, saturated or unsaturated, branched or cyclic, hydrofluoro- or perfluoroalkyl radical;
      it being possible for the hydrocarbon-based chain constituting said radicals to be, where appropriate, interrupted with 1, 2, 3 or more heteroatoms chosen from:
an oxygen,
a sulfur,
a nitrogen, and
a silicon,
   and it being possible for it to be optionally substituted with at least one radical chosen from:
—OR₄,
—SR₄,
—NR₄R₅,
—COOR₄,
—CONHR₄,
—CN,
a halogen atom,
a C₁ to C₆ hydrofluoro- or perfluoroalkyl radical and/or
a C₃ to C₈ cycloalkyl radical,
   with R₄ and R₅ possibly representing, independently of one another, a hydrogen atom, or a C₁ to C₃₀, in particular C₁ to C₁₂, saturated, or C₂ to C₃₀, in particular C₂ to CO₂, unsaturated, linear, or C₃ to C₃₀, in particular C₃ to C₁₂, saturated or unsaturated, branched or cyclic, alkyl, perfluoroalkyl or hydrofluoroalkyl radical; or a C₆ to C₁₀ aryl radical,
X represents a radical chosen from the groups:

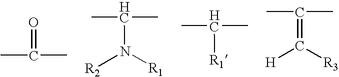

with R₁, R₂ and R₃ representing, independently of one another, a hydrogen atom, or a radical R, with R as defined above, and R′₁ represents a hydrogen atom, an —OH group or a radical R as defined above, R₁ also possibly denoting a C₆ to C₁₀ aryl radical;
S represents a monosaccharide or a polysaccharide comprising up to 20 sugar units, in particular up to 6 sugar units, in pyranose and/or furanose form of the L and/or D series, it being possible for said monosaccharide or polysaccharide to be substituted with a hydroxyl group which is necessarily free, and optionally one or more optionally protected amine function(s), and
the S—CH₂—X bond represents a bond of C-anomeric nature which may be α or β,
   and also the cosmetically acceptable salts thereof, the solvates thereof, such as the hydrates, and the isomers thereof.

In the context of the present invention, the term “halogen” is intended to mean chlorine, fluorine, bromine or iodine.

The term “aryl” denotes an aromatic ring such as phenyl, optionally substituted with one or more C₁-C₄ alkyl radicals.

The term “C₃ to C₈ cycloalkyl” denotes an aliphatic ring containing from 3 to 8 carbon atoms, including, for example, cyclopropyl, cyclopentyl and cyclohexyl.

Among the alkyl groups that are suitable for implementing the invention, mention may in particular be made of methyl, ethyl, isopropyl, n-propyl, n-butyl, t-butyl, isobutyl, sec-butyl, pentyl, n-hexyl, cyclopropyl, cyclopentyl, cyclohexyl and allyl groups.

According to one embodiment of the invention, use may be made of a C-glycoside derivative corresponding to formula (I) for which S may represent a monosaccharide or a polysaccharide containing up to 6 sugar units, in pyranose and/or furanose form and of the L and/or D series, said monosaccharide or polysaccharide having at least one hydroxyl function that is necessarily free and/or optionally one or more amine functions that is (are) necessarily protected, X and R conserving, moreover, all the definitions given above.

Advantageously, a monosaccharide of the invention may be chosen from D-glucose, D-galactose, D-mannose, D-xylose, D-lyxose, L-fucose, L-arabinose, L-rhamnose, D-glucuronic acid, D-galacturonic acid, D-iduronic acid, N-acetyl-D-glucosamine and N-acetyl-D-galactosamine, and advantageously denotes D-glucose, D-xylose, N-acetyl-D-glucosamine or L-fucose, and in particular D-xylose.

More particularly, a polysaccharide of the invention containing up to 6 sugar units may be chosen from D-maltose, D-lactose, D-cellobiose, D-maltotriose, a disaccharide combining a uronic acid chosen from D-iduronic acid and D-glucuronic acid with a hexosamine chosen from D-galactosamine, D-glucosamine, N-acetyl-D-galactosamine and N-acetyl-D-glucosamine, and an oligosaccharide containing at least one xylose which may be advantageously chosen from xylobiose, methyl-β-xylobioside, xylotriose, xylotetraose, xylopentaose and xylohexaose, and in particular the xylobiose which is composed of two xylose molecules linked by a 1-4 linkage.

More particularly, S may represent a monosaccharide chosen from D-glucose, D-xylose, L-fucose, D-galactose and D-maltose, and in particular D-xylose.

According to another embodiment of the invention, use may be made of C-glycoside derivatives corresponding to formula (I) for which X represents a group chosen from —CO—, —CH(OH)—, —CH(NR₁R₂)— and —CH(R)—, in particular —CO—, —CH(OH)—, —CH(NH₂)—, —CH(NHCH₂CH₂CH₂OH)—, —CH(NHPh)—, —CH(CH₃)—, and more particularly a —CO—, —CH(OH)— or —CH(NH₂)— group, and preferably a —CH(OH)— group, S and R conserving, moreover, all the definitions given above.

According to another embodiment of the invention, use may be made of a C-glycoside derivative corresponding to formula (I) for which R represents a C₁ to C₂₀, in particular C₁ to C₁₀, saturated or C₂ to C₂₀, in particular C₂ to C₁₀, unsaturated linear alkyl radical, or a C₃ to C₂₀, in particular C₃ to C₁₀, saturated or unsaturated, branched or cyclic alkyl radical, optionally substituted as described above, S and X conserving, moreover, all the definitions given above. Preferably, R denotes a C₁-C₄, in particular C₁-C₃, linear radical optionally substituted with —OH, —COOH or —COOR₁₂, R″₂ being a C₁-C₄ saturated alkyl radical, in particular ethyl.

Preferably, R denotes an unsubstituted C₁-C₄, in particular C₁-C₂, linear alkyl radical, in particular ethyl.

Among the C-glycoside derivatives of formula (I), use is preferably made of those for which:
R represents a C₁ to C₂₀, in particular C₁ to C₁₀, saturated or C₂ to C₂₀, in particular C₂ to C₁₀, unsaturated linear alkyl radical, or a C₃ to C₂₀, in particular C₃ to C₁₀, saturated or unsaturated, branched or cyclic alkyl radical, optionally substituted as described above;
S represents a monosaccharide as described above;
X represents —CO—, —CH(OH)—, —CH(NR₁R₂)— or —CH(R)—as described above.

Preferably, use is made of a C-glycoside derivative of formula (I) for which:
R denotes a C₁-C₄, in particular C₁-C₃, linear radical optionally substituted with —OH, —COOH or —COOR₁₂, R₁₂ being a C₁-C₄ saturated alkyl radical, in particular ethyl;
S represents a monosaccharide as described above;
X represents a group chosen from —CO—, —CH(OH)—, —CH(NH₂)—, —CH(NHCH₂CH₂CH₂OH)—, CH(NHPh)- and —CH(CH₃)—, and more particularly a —CO—, —CH(OH)— or —CH(NH₂)— group, and preferably a —CH(OH)— group.

Preferably, use is made of a C-glycoside derivative of formula (I) for which:
R denotes an unsubstituted C₁-C₄, in particular C₁-C₂, linear alkyl radical, in particular ethyl,
S represents a monosaccharide as described above; especially D-glucose, D-xylose, N-acetyl-D-glucosamine or L-fucose, and in particular D-xylose;
X represents a group chosen from —CO—, —CH(OH)— and —CH(NH₂)—, and preferably a —CH(OH)— group.

The salts that are acceptable for the nontherapeutic use of the compounds described in the present invention comprise conventional nontoxic salts of said compounds, such as those formed from organic or inorganic acids. By way of example, mention may be made of the salts of mineral acids, such as sulfuric acid, hydrochloric acid, hydrobromic acid, hydroiodic acid, phosphoric acid or boric acid. Mention may also be made of the salts of organic acids, which may comprise one or more carboxylic, sulfonic or phosphonic acid groups. They may be linear, branched or cyclic aliphatic acids or else aromatic acids. These acids may also comprise one or more heteroatoms chosen from O and N, for example in the form of hydroxyl groups. Mention may in particular be made of propionic acid, acetic acid, terephthalic acid, citric acid and tartaric acid.

When the compound of formula (I) comprises an acid group, the neutralization of the acid group(s) can be carried out with a mineral base, such as LiOH, NaOH, KOH, Ca(OH)₂, NH₄OH, Mg(OH)₂ or Zn(OH)₂; or with an organic base such as a primary, secondary or tertiary alkylamine, for example triethylamine or butylamine. This primary, secondary or tertiary alkylamine may comprise one or more nitrogen and/or oxygen atoms and may therefore comprise, for example, one or more alcohol functions; mention may be made of 2-amino-2-methylpropanol, triethanolamine, 2-(dimethylamino)propanol or 2-amino-2-(hydroxymethyl)-1,3-propanediol. Mention may also be made of lysine or 3-(dimethylamino)propylamine.

The solvates that are acceptable for the compounds described in the present invention comprise conventional solvates, such as those formed during the final stage of preparation of said compounds due to the presence of solvents. By way of example, mention may be made of the solvates due to the presence of water or of linear or branched alcohols such as ethanol or isopropanol.

Among the C-glycoside derivatives of formula (I) used according to the invention, the following are most particularly considered:
1. C-β-D-xylopyranoside-n-propan-2-one;
2. C-α-D-xylopyranoside-n-propan-2-one;
3. 1-[2-(3-hydroxypropylamino)propyl]-C-β-D-xylopyranose;
4. 1-[2-(3-hydroxypropylamino)propyl]-C-α-D-xylopyranose;
5. C-β-D-xylopyranoside-2-hydroxypropane;
6. C-α-D-xylopyranoside-2-hydroxypropane;
7. C-β-D-xylopyranoside-2-aminopropane;
8. C-α-D-xylopyranoside-2-aminopropane;
9. C-β-D-xylopyranoside-2-(phenylamino)propane;
10. C-α-D-xylopyranoside-2-(phenylamino)propane;
11. 3-methyl-4-(C-β-D-xylopyranoside)butyric acid ethyl ester;
12. 3-methyl-4-(C-α-D-xylopyranoside)butyric acid ethyl ester;
13. 6-(C-β-D-xylopyranoside)-5-ketohexanoic acid;
14. 6-(C-α-D-xylopyranoside)-5-ketohexanoic acid;
15. 6-(C-β-D-xylopyranoside)-5-hydroxyhexanoic acid;
16. 6-(C-α-D-xylopyranoside)-5-hydroxyhexanoic acid;
17. 6-(C-β-D-xylopyranoside)-5-aminohexanoic acid;
18. 6-(C-α-D-xylopyranoside)-5-aminohexanoic acid;
19. 6-(C-β-D-xylopyranoside)-5-(phenylamino)hexanoic acid;
20. 6-(C-α-D-xylopyranoside)-5-(phenylamino)hexanoic acid;
21. 1-(C-β-D-xylopyranoside)hexane-2,6-diol;
22. 1-(C-α-D-xylopyranoside)hexane-2,6-diol;
23. 5-(C-β-D-xylopyranoside)-4-ketopentanoic acid;
24. 5-(C-α-D-xylopyranoside)-4-ketopentanoic acid;
25. 5-(C-β-D-xylopyranoside)-4-hydroxypentanoic acid;
26. 5-(C-α-D-xylopyranoside)-4-hydroxypentanoic acid;
27. 5-(C-β-D-xylopyranoside)-4-aminopentanoic acid;
28. 5-(C-α-D-xylopyranoside)-4-aminopentanoic acid;
29. 5-(C-β-D-xylopyranoside)-4-(phenylamino)pentanoic acid;
30. 5-(C-α-D-xylopyranoside)-4-(phenylamino)pentanoic acid;
31. 1-(C-β-D-xylopyranoside)pentane-2,5-diol;
32. 1-(C-α-D-xylopyranoside)pentane-2,5-diol;
33. 1-(C-β-D-fucopyranoside)propan-2-one;
34. 1-(C-α-D-fucopyranoside)propan-2-one;
35. 1-(C-β-L-fucopyranoside)propan-2-one;
36. 1-(C-α-L-fucopyranoside)propan-2-one;
37. 1-(C-β-D-fucopyranoside)-2-hydroxypropane;
38. 1-(C-α-D-fucopyranoside)-2-hydroxypropane;
39. 1-(C-β-L-fucopyranoside)-2-hydroxypropane;
40. 1-(C-α-L-fucopyranoside)-2-hydroxypropane;
41. 1-(C-β-D-fucopyranoside)-2-aminopropane;
42. 1-(C-α-D-fucopyranoside)-2-aminopropane;
43. 1-(C-β-L-fucopyranoside)-2-aminopropane;
44. 1-(C-α-L-fucopyranoside)-2-aminopropane;
45. 1-(C-β-D-fucopyranoside)-2-(phenylamino)propane;
46. 1-(C-α-D-fucopyranoside)-2-(phenylamino)propane;
47. 1-(C-β-L-fucopyranoside)-2-(phenylamino)propane;
48. 1-(C-α-L-fucopyranoside)-2-(phenylamino)propane;
49. 3-methyl-4-(C-β-D-fucopyranoside)butyric acid ethyl ester;
50. 3-methyl-4-(C-α-D-fucopyranoside)butyric acid ethyl ester;
51. 3-methyl-4-(C-β-L-fucopyranoside)butyric acid ethyl ester;
52. 3-methyl-4-(C-α-L-fucopyranoside)butyric acid ethyl ester;
53. 6-(C-β-D-fucopyranoside)-5-ketohexanoic acid;
54. 6-(C-α-D-fucopyranoside)-5-ketohexanoic acid;
55. 6-(C-β-L-fucopyranoside)-5-ketohexanoic acid;
56. 6-(C-α-L-fucopyranoside)-5-ketohexanoic acid;
57. 6-(C-β-D-fucopyranoside)-5-hydroxyhexanoic acid;
58. 6-(C-β-D-fucopyranoside)-5-hydroxyhexanoic acid;
59. 6-(C-β-L-fucopyranoside)-5-hydroxyhexanoic acid;
60. 6-(C-α-L-fucopyranoside)-5-hydroxyhexanoic acid;
61. 6-(C-β-D-fucopyranoside)-5-aminohexanoic acid;
62. 6-(C-α-D-fucopyranoside)-5-aminohexanoic acid;
63. 6-(C-β-L-fucopyranoside)-5-aminohexanoic acid;
64. 6-(C-α-L-fucopyranoside)-5-aminohexanoic acid;
65. 1-(C-β-D-fucopyranoside)hexane-2,6-diol;
66. 1-(C-α-D-fucopyranoside)hexane-2,6-diol;
67. 1-(C-β-L-fucopyranoside)hexane-2,6-diol;
68. 1-(C-α-L-fucopyranoside)hexane-2,6-diol;
69. 5-(C-β-D-fucopyranoside)-4-ketopentanoic acid;
70. 5-(C-α-D-fucopyranoside)-4-ketopentanoic acid;
71. 5-(C-β-L-fucopyranoside)-4-ketopentanoic acid;
72. 5-(C-α-L-fucopyranoside)-4-ketopentanoic acid;
73. 5-(C-β-D-fucopyranoside)-4-hydroxypentanoic acid;
74. 5-(C-α-D-fucopyranoside)-4-hydroxypentanoic acid;
75. 5-(C-β-L-fucopyranoside)-4-hydroxypentanoic acid;
76. 5-(C-α-L-fucopyranoside)-4-hydroxypentanoic acid;
77. 5-(C-β-D-fucopyranoside)-4-aminopentanoic acid;
78. 5-(C-α-D-fucopyranoside)-4-aminopentanoic acid;
79. 5-(C-β-L-fucopyranoside)-4-aminopentanoic acid;
80. 5-(C-α-L-fucopyranoside)-4-aminopentanoic acid;
81. 1-(C-β-D-fucopyranoside)pentane-2,5-diol;
82. 1-(C-α-D-fucopyranoside)pentane-2,5-diol;
83. 1-(C-β-L-fucopyranoside)pentane-2,5-diol;
84. 1-(C-α-L-fucopyranoside)pentane-2,5-diol;
85. 1-(C-β-D-glucopyranosyl)-2-hydroxypropane;
86. 1-(C-α-D-glucopyranosyl)-2-hydroxypropane;
87. 1-(C-β-D-glucopyranosyl)-2-aminopropane;
88. 1-(C-α-D-glucopyranosyl)-2-aminopropane;
89. 1-(C-β-D-glucopyranosyl)-2-(phenylamino)propane;
90. 1-(C-α-D-glucopyranosyl)-2-(phenylamino)propane;
91. 3-methyl-4-(C-1-D-glucopyranosyl)butyric acid ethyl ester;
92. 3-methyl-4-(C-α-D-glucopyranosyl)butyric acid ethyl ester;
93. 6-(C-β-D-glucopyranosyl)-5-ketohexanoic acid;
94. 6-(C-α-D-glucopyranosyl)-5-ketohexanoic acid;
95. 6-(C-β-D-glucopyranosyl)-5-hydroxyhexanoic acid;
96. 6-(C-α-D-glucopyranosyl)-5-hydroxyhexanoic acid;
97. 6-(C-β-D-glucopyranosyl)-5-aminohexanoic acid;
98. 6-(C-α-D-glucopyranosyl)-5-aminohexanoic acid;
99. 6-(C-β-D-glucopyranosyl)-5-(phenylamino)hexanoic acid;
100. 6-(C-α-D-glucopyranosyl)-5-(phenylamino)hexanoic acid;
101. 1-(C-β-D-glucopyranosyl)hexane-2,6-diol;
102. 1-(C-α-D-glucopyranosyl)hexane-2,6-diol;
103. 6-(C-β-D-glucopyranosyl)-5-ketopentanoic acid;
104. 6-(C-α-D-glucopyranosyl)-5-ketopentanoic acid;
105. 6-(C-β-D-glucopyranosyl)-5-hydroxypentanoic acid;
106. 6-(C-α-D-glucopyranosyl)-5-hydroxypentanoic acid;
107. 6-(C-β-D-glucopyranosyl)-5-aminopentanoic acid;
108. 6-(C-α-D-glucopyranosyl)-5-hydroxypentanoic acid;
109. 6-(C-β-D-glucopyranosyl)-5-(phenylamino)pentanoic acid;
110. 6-(C-α-D-glucopyranosyl)-5-(phenylamino)pentanoic acid;
111. 1-(C-β-D-glucopyranosyl)pentane-2,5-diol;
112. 1-(C-α-D-glucopyranosyl)pentane-2,5-diol;
113. 1-(C-β-D-galactopyranosyl)-2-hydroxypropane;
114. 1-(C-α-D-galactopyranosyl)-2-hydroxypropane;
115. 1-(C-β-D-galactopyranosyl)-2-aminopropane;
116. 1-(C-β-D-galactopyranosyl)-2-aminopropane;
117. 1-(C-β-D-galactopyranosyl)-2-(phenylamino)propane;
118. 1-(C-α-D-galactopyranosyl)-2-(phenylamino)propane;
119. 3-methyl-4-(β-D-galactopyranosyl)butyric acid ethyl ester;
120. 3-methyl-4-(α-D-galactopyranosyl)butyric acid ethyl ester;
121. 6-(C-β-D-galactopyranosyl)-5-ketohexanoic acid;
122. 6-(C-α-D-galactopyranosyl)-5-ketohexanoic acid;
123. 6-(C-1-D-galactopyranosyl)-5-hydroxyhexanoic acid;
124. 6-(C-α-D-galactopyranosyl)-5-hydroxyhexanoic acid;
125. 6-(C-β-D-galactopyranosyl)-5-aminohexanoic acid;
126. 6-(C-α-D-galactopyranosyl)-5-aminohexanoic acid;
127. 6-(C-β-D-galactopyranosyl)-5-(phenylamino)hexanoic acid;
128. 6-(C-α-D-galactopyranosyl)-5-(phenylamino)hexanoic acid;
129. 1-(C-β-D-galactopyranosyl)hexane-2,6-diol;
130. 1-(C-α-D-galactopyranosyl)hexane-2,6-diol;
131. 6-(C-β-D-galactopyranosyl)-5-ketopentanoic acid;
132. 6-(C-α-D-galactopyranosyl)-5-ketopentanoic acid;
133. 6-(C-β-D-galactopyranosyl)-5-hydroxypentanoic acid;
134. 6-(C-α-D-galactopyranosyl)-5-hydroxypentanoic acid;
135. 6-(C-β-D-galactopyranosyl)-5-aminopentanoic acid;
136. 6-(C-α-D-galactopyranosyl)-5-aminopentanoic acid;
137. 6-(C-β-D-galactopyranosyl)-5-(phenylamino)pentanoic acid;
138. 6-(C-α-D-galactopyranosyl)-5-(phenylamino)pentanoic acid;
139. 1-(C-β-D-galactopyranosyl)pentane-2,6-diol;
140. 1-(C-α-D-galactopyranosyl)pentane-2,6-diol;
141. 1-(C-β-D-fucofuranosyl)propan-2-one;
142. 1-(C-α-D-fucofuranosyl)propan-2-one;
143. 1-(C-β-L-fucofuranosyl)propan-2-one;
144. 1-(C-α-L-fucofuranosyl)propan-2-one;
145. 3′-(acetamido-C-β-D-glucopyranosyl)propan-2′-one;
146. 3′-(acetamido-C-α-D-glucopyranosyl)propan-2′-one;
147. 1-(C-β-D-glucopyranosyl)-2-hydroxypropane;
148. 1-(C-β-D-glucopyranosyl)-2-aminopropane;
149. 1-(acetamido-C-β-D-glucopyranosyl)-2-(phenylamino)propane;
150. 1-(acetamido-C-α-D-glucopyranosyl)-2-(phenylamino)propane;
151. 3-methyl-4-(acetamido-C-β-D-glucopyranosyl)butyric acid ethyl ester;
152. 3-methyl-4-(acetamido-C-α-D-glucopyranosyl)butyric acid ethyl ester;
153. 6-(acetamido-C-β-D-glucopyranosyl)-5-ketohexanoic acid;
154. 6-(acetamido-C-α-D-glucopyranosyl)-5-ketohexanoic acid;
155. 6-(acetamido-C-β-D-glucopyranosyl)-5-hydroxyhexanoic acid;
156. 6-(acetamido-C-α-D-glucopyranosyl)-5-hydroxyhexanoic acid;
157. 6-(acetamido-C-β-D-glucopyranosyl)-5-aminohexanoic acid;
158. 6-(acetamido-C-α-D-glucopyranosyl)-5-aminohexanoic acid;
159. 6-(acetamido-C-β-D-glucopyranosyl)-5-(phenylamino)hexanoic acid;
160. 6-(acetamido-C-α-D-glucopyranosyl)-5-(phenylamino)hexanoic acid;
161. 1-(acetamido-C-β-D-glucopyranosyl)hexane-2,6-diol;
162. 1-(acetamido-C-α-D-glucopyranosyl)hexane-2,6-diol;
163. 6-(acetamido-C-β-D-glucopyranosyl)-5-ketopentanoic acid;
164. 6-(acetamido-C-α-D-glucopyranosyl)-5-ketopentanoic acid;
165. 6-(acetamido-C-β-D-glucopyranosyl)-5-hydroxypentanoic acid;
166. 6-(acetamido-C-α-D-glucopyranosyl)-5-hydroxypentanoic acid;
167. 6-(acetamido-C-β-D-glucopyranosyl)-5-aminopentanoic acid;
168. 6-(acetamido-C-α-D-glucopyranosyl)-5-aminopentanoic acid;
169. 6-(acetamido-C-β-D-glucopyranosyl)-5-(phenylamino)pentanoic acid;
170. 6-(acetamido-C-α-D-glucopyranosyl)-5-(phenylamino)pentanoic acid;
171. 1-(acetamido-C-β-D-glucopyranosyl)pentane-2,5-diol;
172. 1-(acetamido-C-α-D-glucopyranosyl)pentane-2,5-diol.

By way of nonlimiting illustration of the C-glycoside derivatives most particularly suitable for the invention, mention may in particular be made of the following derivatives:
C-β-D-xylopyranoside-n-propan-2-one,
C-α-D-xylopyranoside-n-propan-2-one,
C-β-D-xylopyranoside-2-hydroxypropane,
C-α-D-xylopyranoside-2-hydroxypropane,
1-(C-β-D-fucopyranoside)propan-2-one,
1-(C-α-D-fucopyranoside)propan-2-one,
1-(C-β-L-fucopyranoside)propan-2-one,
1-(C-α-L-fucopyranoside)propan-2-one,
1-(C-β-D-fucopyranoside)-2-hydroxypropane,
1-(C-α-fucopyranoside)-2-hydroxypropane,
1-(C-β-L-fucopyranoside)-2-hydroxypropane,
1-(C-α-L-fucopyranoside)-2-hydroxypropane,
1-(C-β-D-glucopyranosyl)-2-hydroxypropane,
1-(C-α-D-glucopyranosyl)-2-hydroxypropane,
1-(C-β-D-galactopyranosyl)-2-hydroxypropane,
1-(C-α-D-galactopyranosyl)-2-hydroxypropane,
1-(C-β-D-fucofuranosyl)propan-2-one,
1-(C-α-D-fucofuranosyl)propan-2-one,
1-(C-β-L-fucofuranosyl)propan-2-one,
1-(C-α-L-fucofuranosyl)propan-2-one,
C-β-D-maltopyranoside-n-propan-2-one,
C-α-D-maltopyranoside-n-propan-2-one,
C-β-D-maltopyranoside-2-hydroxypropane,
C-α-D-maltopyranoside-2-hydroxypropane, isomers thereof and mixtures thereof.

According to one embodiment, C-β-D-xylopyranoside-2-hydroxypropane or C-α-D-xylopyranoside-2-hydroxypropane, and better still C-β-D-xylopyranoside-2-hydroxypropane, may be advantageously used for the preparation of a composition according to the invention.

According to a specific embodiment, the C-glycoside derivative may be C-β-D-xylopyranoside-2-hydroxypropane in the form of a solution at 30% by weight with respect to active material in a water/propylene glycol mixture (60/40% by weight) such as the product manufactured by Chimex under the trade name “Mexoryl SBB®”.

Of course, according to the invention, a C-glycoside derivative corresponding to formula (I) may be used alone or as a mixture with other C-glycoside derivatives and in any proportions.

A C-glycoside derivative suitable for the invention may in particular be obtained by the method of synthesis described in document WO 02/051828.

The amount of C-glycoside derivative to be used in a composition according to the invention depends on the desired cosmetic or therapeutic effect, and can therefore vary to a large extent.

Those skilled in the art can, on the basis of their general knowledge, readily determine the appropriate amounts.

A composition according to the invention may comprise a C-glycoside derivative in a proportion of approximately 0.0001% to approximately 25% by weight of active material relative to the total weight of the composition, and in particular of approximately 0.001% to approximately 10% by weight of active material, and more particularly of approximately 0.05% to approximately 5% by weight of active material of C-glycoside derivative, relative to the total weight of the composition.

In the context of the present invention, the ratio by weight of the hyaluronic acid or a derivative thereof to the C-glycoside derivative, in the composition, may range between 0.001 and 1000, in particular between 1 and 10, and most particularly between 2 and 4.

### Galenics

The combination of at least one C-glycoside derivative and of at least one hyaluronic acid or a derivative thereof according to the invention is preferably formulated for topical administration, i.e. containing a cosmetically or dermatologically acceptable medium, i.e. a medium compatible with the skin, the nails, the mucous membranes, the tissues, the scalp and/or the hair.

It is also clear that the effective amount of the active agents corresponds to the amount necessary to obtain the desired result, and the formulation of the compositions according to the invention depends on the use for which these compositions are intended.

In particular, two major categories of topical compositions according to the invention can be distinguished, according to the conditions under which they will be applied to the skin.

The first category corresponds to cosmetic compositions, i.e. compositions intended to be applied to normal skin in order to improve the esthetics and in particular the comfort thereof. Normal skin is defined by the absence of pathological conditions such as infections, an inflammation, erythema or injuries such as a burn or a cut. However, in this definition, normal skin does not signify skin in perfect condition.

In particular, normal skin may show signs of dryness which may be of exogenous origin (the skin becomes dry, for example, during exposure to dry and very cold air), or of endogenous physiological origin (for example, at the time of the decrease in hormone levels associated with the menopause).

The cosmetic composition is thus formulated to be applied to the skin and/or its appendages and comprises at least one hyaluronic acid or a derivative thereof and at least one C-glycoside derivative, said composition making it possible to improve the barrier function.

According to the invention, the combination of the C-glycoside derivative and of hyaluronic acid or a derivative thereof may also be used for the preparation of a therapeutic, more specifically dermatological, composition for improving the condition of damaged skin, such as skin requiring tissue repair and/or regeneration at the level of the dermis, of the epidermis and of the dermal-epidermal junction.

The damage under consideration may be of any origin, for example infectious, allergic, nerve-related or traumatic. A therapeutic composition may be applied directly to the damaged site or to the area surrounding it.

Thus, the invention also relates to the use of the combination of at least one C-glycoside derivative and of at least one hyaluronic acid or of at least one derivative thereof, for the preparation of a topical composition formulated for therapeutic use, for example on damaged skin such as skin requiring tissue repair at the level of the dermis, of the epidermis and of the dermal-epidermal junction.

The composition will then be formulated with a pharmaceutically acceptable carrier.

According to a preferred embodiment of the invention, the composition has a pH close to that of the skin, between 4 and 7.

When it is applied topically, the composition according to the invention may be applied to the face, the neck, the scalp, the mucous membranes and the nails or any other cutaneous area of the body, including the hands and the feet.

The term “physiologically acceptable medium” is intended to denote a medium compatible with the skin and/or its appendages or keratin materials and/or fibers of human beings, for instance, and in a nonlimiting manner, the skin, the mucous membranes, the nails, the scalp and/or the hair.

This physiologically acceptable medium comprises water, optionally as a mixture with one or more organic solvents such as C₁-C₈ alcohols, in particular ethanol, isopropanol, tert-butanol, n-butanol, polyols such as glycerol, propylene glycol or butylene glycol, and polyol ethers.

The composition is in particular in the form of aqueous, aqueous-alcoholic or oily solutions, dispersions of the lotion or serum type, anhydrous or oily gels, emulsions of liquid or semi-liquid consistency of the milk type, obtained by dispersion of a fatty phase in an aqueous phase (O/W) or vice versa (W/O), multiple emulsions (triple: W/O/W or O/W/O), suspensions or emulsions of soft, semi-solid or solid consistency of the cream or gel type, in particular aqueous cream or gel type, microemulsions, dispersions of a fatty phase in an aqueous phase by means of polymeric nanoparticles such as nanospheres and nanocapsules, or lipid vesicles of ionic and/or nonionic type (liposomes, niosomes, oleosomes), nanoemulsions, or thin films, or else microcapsules or microparticles. A composition according to the invention may also be used in the form of a dual water and oil phase.

The amounts of the various constituents of the compositions used according to the invention are those conventionally used in the fields under consideration.

These compositions are prepared according to the usual methods.

In addition, the compositions used according to the invention may be more or less fluid and may have the appearance of a white or colored cream, an ointment, a milk, a lotion, a serum, a paste or a foam. They may optionally be applied to the skin in the form of an aerosol. They may also be in solid form, for example in the form of a stick.

According to a specific embodiment of the invention, the composition is in the form of a transparent gel. In particular, this transparent gel is characterized in that it is obtained by combining the active agents with a homopolymer of a monomer comprising a sulfonic group. More particularly, said gel contains from 0 to 1% of oil.

The polymers comprising at least one monomer comprising a sulfonic group, used in this type of transparent gel, are advantageously water-soluble or water-dispersible or water-swellable. The polymers used for this type of transparent gel are homopolymers which can be obtained from at least one ethylenically unsaturated monomer comprising a sulfonic group, which may be in free form or partially or completely neutralized form.

Preferably, the polymers used for this type of transparent gel may be partially or completely neutralized with a mineral base (sodium hydroxide, potassium hydroxide, aqueous ammonia) or an organic base such as mono-, di- or triethanolamine, an aminomethylpropanediol, N-methylglucamine, basic amino acids such as arginine and lysine, and mixtures of these compounds. They are generally neutralized. In the present invention, the term “neutralized” is intended to mean polymers that are completely or almost completely neutralized, i.e. at least 90% neutralized.

These polymers generally have a number-average molecular weight ranging from 1000 to 20 000 000 g/mol, preferably ranging from 20 000 to 5 000 000, and even more preferably from 100 000 to 1 500 000 g/mol.

These polymers according to the invention may be crosslinked or noncrosslinked.

The monomers comprising a sulfonic group, of the polymer, used in these transparent gels are chosen in particular from vinylsulfonic acid, styrenesulfonic acid, (meth)acrylamido(C₁-C₂₂)alkylsulfonic acids, N—(C₁-C₂₂)alkyl(meth) acrylamido (C₁-C₂₂) alkylsulfonic acids, such as undecylacrylamidomethanesulfonic acid, and also the partially or completely neutralized forms thereof, and mixtures thereof.

According to a preferred embodiment of the invention, the monomers comprising a sulfonic group are chosen from (meth)acrylamido(C₁-C₂₂)alkylsulfonic acids such as, for example, acrylamidomethanesulfonic acid, acrylamidoethanesulfonic acid, acrylamidopropanesulfonic acid, 2-acrylamido-2-methylpropanesulfonic acid, 2-methacrylamido-2-methylpropanesulfonic acid, 2-acrylamido-n-butanesulfonic acid, 2-acrylamido-2,4,4-trimethylpentanesulfonic acid, 2-methacrylamidododecylsulfonic acid and 2-acrylamido-2,6-dimethyl-3-heptanesulfonic acid, and also the partially or completely neutralized forms thereof, and mixtures thereof.

More particularly, use is made of 2-acrylamido-2-methylpropanesulfonic acid (AMPS) and also the partially or completely neutralized forms thereof.

By way of other polymers suitable for this type of gel, mention may in particular be made of the crosslinked and neutralized homopolymer of 2-acrylamido-2-methylpropanesulfonic acid sold by the company Clariant under the trade name “Hostacerin® AMPS” (CTFA name: ammonium polyacryldimethyltauramide, INCI name, Ammonium polyacryloyl dimethyl taurate).

The homopolymer of a monomer comprising a sulfonic group may be present in a transparent gel at a content with respect to active material ranging, for example, from 0.05% to 5% by weight, preferably ranging from 0.1% to 5% by weight, preferentially ranging from 0.05% to 2% by weight, for example from 0.1% to 4% by weight, in particular 0.25% by weight, relative to the total weight of the composition.

When the composition used according to the invention, irrespective of its nature, comprises an oily phase, the latter preferably contains at least one oil. It may also contain other fatty substances.

As oils that can be used in the composition of the invention, mention may be made, for example, of:
hydrocarbon-based oils of animal origin, such as perhydrosqualene;
hydrocarbon-based oils of plant origin, such as liquid triglycerides of fatty acids comprising from 4 to 10 carbon atoms, such as heptanoic or octanoic acid triglycerides or else, for example, sunflower oil, maize oil, soybean oil, marrow oil, grapeseed oil, sesame oil, hazelnut oil, apricot oil, macadamia oil, arara oil, sunflower oil, castor oil and avocado oil, caprylic/capric acid triglycerides such as those sold by the company Stearineries Dubois or those sold under the names Miglyol 810, 812 and 818 by the company Dynamit Nobel, jojoba oil, and shea butter oil;
synthetic esters and ethers, in particular of fatty acids, such as oils of formulae R¹COOR² and R¹OR² in which R¹ represents the residue of a fatty acid comprising from 8 to 29 carbon atoms, and R² represents a branched or unbranched hydrocarbon-based chain containing from 3 to 30 carbon atoms, such as, for example, purcellin oil, isononyl isononanoate, isopropyl myristate, 2-ethylhexyl palmitate, 2-octyldodecyl stearate, 2-octyldodecyl erucate, isostearyl isostearate; hydroxylated esters such as isostearyl lactate, octyl hydroxystearate, octyldodecyl hydroxystearate, diisostearyl malate, triisocetyl citrate, heptanoates, octanoates and decanoates of fatty alcohols; polyol esters, such as propylene glycol dioctanoate, neopentyl glycol diheptanoate and diethylene glycol diisononanoate; and pentaerythritol esters such as pentaerythrityl tetraisostearate;
linear or branched hydrocarbons of mineral or synthetic origin, such as volatile or nonvolatile liquid paraffins, and derivatives thereof, petroleum jelly, polydecenes, hydrogenated polyisobutene such as parleam oil;
fatty alcohols containing from 8 to 26 carbon atoms, such as cetyl alcohol, stearyl alcohol and their blend (cetylstearyl alcohol), octyldodecanol, 2-butyloctanol, 2-hexyldecanol, 2-undecylpentadecanol, oleyl alcohol or linoleyl alcohol;
partially hydrocarbon-based and/or silicone-based fluoro oils such as those described in document JP-A-2-295912;
silicone oils such as volatile or nonvolatile dipolydimethylsiloxanes (PDMS) having a linear or cyclic silicone-based chain, which are liquid or pasty at ambient temperature, in particular cyclopolydimethylsiloxanes (cyclomethicones) such as cyclohexasiloxane; polydimethylsiloxanes comprising alkyl, alkoxy or phenyl groups, which are pendent or at the end of the silicone-based chain, groups containing from 2 to 24 carbon atoms; phenylsilicones, such as phenyl trimethicones, phenyl dimethicones, phenyltrimethylsiloxydiphenylsiloxanes, diphenyl dimethicones, diphenylmethyldiphenyltrisiloxanes, 2-phenylethyltrimethylsiloxysilicates, and polymethylphenylsiloxanes;
and mixtures thereof.

The term “hydrocarbon-based oil” in the abovementioned list of oils is intended to mean any oil comprising predominantly carbon and hydrogen atoms, and optionally ester, ether, fluoro, carboxylic acid and/or alcohol groups.

The other fatty substances that may be present in the oily phase are, for example, fatty acids containing from 8 to 30 carbon atoms, such as stearic acid, lauric acid, palmitic acid and oleic acid; waxes such as lanolin, beeswax, carnauba wax and candelilla wax, paraffin and lignite waxes or microcrystalline waxes, ceresin or ozokerite, synthetic waxes such as polyethylene waxes, Fischer-Tropsch waxes; silicone resins such as trifluoromethyl-C₁-4-alkyl dimethicone and trifluoropropyl dimethicone; and silicone elastomers such as the products sold under the name “KSG” by the company Shin-Etsu, under the names “Trefil”, “BY29” or “EPSX” by the company Dow Corning or under the name “Gransil” by the company Grant Industries.

These fatty substances may be chosen in a varied manner by those skilled in the art in order to prepare a composition having the desired properties, for example of consistency or of texture.

According to a specific embodiment of the invention, the composition according to the invention is a water-in-oil (W/O) or oil-in-water (O/W) emulsion. The proportion of the oily phase of the emulsion may range from 5% to 80% by weight, and preferably from 5% to 50% by weight, relative to the total weight of the composition.

The emulsions generally contain at least one emulsifier chosen from amphoteric, anionic, cationic or nonionic emulsifiers, used alone or as a mixture, and optionally a coemulsifier. The emulsifiers are chosen in a suitable manner according to the emulsion to be obtained (W/O or O/W). The emulsifier and the coemulsifier are generally present in the composition in an amount ranging from 0.3% to 30% by weight, and preferably from 0.5% to 20% by weight, relative to the total weight of the composition.

For W/O emulsions, as emulsifiers, mention may, for example, be made of dimethicone copolyols such as the mixture of cyclomethicone and dimethicone copolyol, sold under the name “DC 5225 C” by the company Dow Corning, and alkyl dimethicone copolyols such as the lauryl methicone copolyol sold under the name “Dow Corning 5200 Formulation Aid” by the company Dow Corning and the cetyl dimethicone copolyol sold under the name Abil EM 90^{R} by the company Goldschmidt.

It is also possible to use, as surfactant for W/O emulsions, a crosslinked solid organopolysiloxane elastomer comprising at least one oxyalkylenated group, such as those obtained according to the procedure in examples 3, 4 and 8 of document U.S. Pat. No. 5,412,004 and in the examples of document U.S. Pat. No. 5,811,487, in particular the product of example 3 (synthesis example) of U.S. Pat. No. 5,412,004, and such as that sold under the reference KSG 21 by the company Shin Etsu. Other types of KSGs sold by the company Shin Etsu may also be used, such as KSG-16.

For O/W emulsions, as emulsifiers, mention may, for example, be made of nonionic emulsifiers such as oxyalkylenated (more particularly polyoxyethylenated) glycerol fatty acid esters; oxyalkylenated sorbitan fatty acid esters; oxyalkylenated (oxyethylenated and/or oxypropylenated) fatty acid esters; oxyalkylenated (oxyethylenated and/or oxypropylenated) fatty alcohol ethers; sugar esters such as sucrose stearate; and mixtures such as the mixture of glyceryl stearate and PEG-40 stearate.

In a known manner, the cosmetic or dermatological composition of the invention may also contain adjuvants that are customary in the cosmetics or dermatological field, such as hydrophilic or lipophilic gelling agents, preservatives, solvents, fragrances, fillers, UV screens, bactericides, odor absorbers, dyestuffs, plant extracts, salts, antioxidants, basic agents, acids, and nonionic, anionic or cationic surfactants.

The amounts of these various adjuvants are those conventionally used in the field under consideration, and for example from 0.01% to 20% of the total weight of the composition. Depending on their nature, these adjuvants may be introduced into the fatty phase, into the aqueous phase and/or into lipid vesicles.

As fillers which can be used in the composition of the invention, mention may, for example, be made, in addition to pigments, of silica powder, an amorphous colloidal silica; talc; polyamide particles, and in particular those sold under the name Orgasol by the company Atochem; polyethylene powders; microspheres based on acrylic copolymers, such as those made of ethylene glycol dimethacrylate/lauryl methacrylate copolymer, sold by the company Dow Corning under the name Polytrap; expanded powders such as hollow microspheres, and in particular the microspheres sold under the name Expancel by the company Kemanord Plast or under the name Micropearl F 80 ED by the company Matsumoto; silicone resin microbeads such as those sold under the name Tospearl by the company Toshiba Silicone; and mixtures thereof. These fillers may be present in amounts ranging from 0 to 20% by weight, and preferably from 1% to 10% by weight, relative to the total weight of the composition.

As hydrophilic or lipophilic gelling agents, mention may in particular be made of carbopols, luvigels, Hostacerin AMPS, Simulgel, acrylamide gelling agents of Sepigel type, such as Sepigel 305® from Seppic, xanthan, guar and cellulose gums, alginates, and mixtures thereof. Mention may also be made of hectorites.

As antioxidants, mention may in particular be made of polyphenols, tannic acid, epigallocatechins and natural extracts containing them, anthocyans, extracts of rosemary, extracts of olive tree leaves, green tea, resveratrol and its derivatives, Pycnogenol, ergothioneine, N-actylcysteine, biotin, chelating agents, idebenone, plant extracts such as Pronalen Bioprotect™ from the company Provital, free-radical scavengers such as vitamin E, coenzyme Q10, bioflavonoids, SODs, phytantriol, lignans, melatonin, pidolates and glutathione.

According to a preferred embodiment of the invention, the composition used according to the invention contains at least one UV screen (or sunscreen) which may be a chemical screen or a physical screen or a mixture of such screens.

By way of illustration and in a nonlimiting manner, mention may be made of the following families (the names correspond to the CTFA screen nomenclature): anthranilates, in particular menthyl anthranilate; benzophenones, in particular benzophenone-1, benzophenone-3, benzophenone-5, benzophenone-6, benzophenone-8, benzophenone-9, benzophenone-12, and preferably benzophenone-2 (oxybenzone), or benzophenone-4 (Uvinul MS40 available from BASF); benzylidenecamphors, in particular 3-benzylidenecamphor, benzylidenecamphorsulfonic acid, camphor benzalkonium methosulfate, polyacrylamidomethylbenzylidenecamphor, terephthalylidenedicamphorsulfonic acid, and preferably 4-methylbenzylidenecamphor (Eusolex 6300 available from Merck); benzimidazoles, in particular benzimidazilate (Neo Heliopan AP available from Haarmann and Reimer), or phenylbenzimidazolesulfonic acid (Eusolex 232 available from Merck); benzotriazoles, in particular drometrizole trisiloxane or methylenebis[(benzotriazolyl)(tetramethylbutyl)phenol] (Tinosorb M available from Ciba); cinnamates, in particular cinoxate, DEA methoxycinnamate, diisopropyl methylcinnamate, glycerylethylhexanoate dimethoxycinnamate, isopropyl methoxycinnamate, isoamyl cinnamate, and preferably ethocrylene (Uvinul N35 available from BASE), octyl methoxycinnamate (Parsol MCX available from Hoffmann La Roche) or octocrylene (Uvinul 539 available from BASF); dibenzoylmethanes, in particular butyl methoxydibenzoylmethane (Parsol 1789); imidazolines, in particular ethylhexyl dimethoxybenzylidene dioxoimidazoline; PABAS, in particular ethyldihydroxypropyl PABA, ethylhexyldimethyl PABA, glyceryl PABA, PABA, PEG-25 PABA, and preferably diethylhexylbutamidotriazone (Uvasorb HEB available from 3V Sigma), ethylhexyltriazone (Uvinul T150 available from BASF), or ethyl PABA (benzocaine); salicylates, in particular dipropylene glycol salicylate, ethylhexyl salicylate, homosalate, or TEA salicylate; triazines, in particular anisotriazine (Tinosorb S available from Ciba); drometrizole trisiloxane, zinc oxide, titanium dioxide, zinc oxide, iron oxide, zirconium oxide and cerium oxide, which may be coated or uncoated.

The amount of screens depends on the desired final use. It may range, for example, from 1% to 20% by weight, and better still from 2% to 10% by weight, relative to the total weight of the composition.

The compositions according to the invention may be applied directly to the skin or, alternatively, to cosmetic or dermatological supports of occlusive or nonocclusive type, intended to be applied locally to the skin.

The support may be an “occlusive” support. By way of example, the support is formed from a thermoplastic, chosen from high- and low-density polyethylenes, polypropylenes, polyvinyl chlorides, ethylene vinyl acetate copolymers, polyesters and polyurethanes, or from a complex of such materials. These materials may also be present in laminated form with at least one metal sheet, such as an aluminum foil.

The support layer may be of any appropriate thickness which will provide the desired support and protective functions. Preferably, the thickness of the support layer is between approximately 20 μm and approximately 1.5 mm. Advantageously, the support layer is sufficiently flexible so as to be able to perfectly fit the profile of the skin, and so as not to cause the user any feeling of discomfort.

Preferably however, the support is “nonocclusive”. In the latter assumption, use is advantageously made of a support that is formed from a paper, a porous or perforated thermoplastic, a woven fabric, a nonwoven or a perforated nonwoven.

According to another embodiment of the invention, said compositions according to the invention may be combined with compositions administered orally, containing additional cosmetic active agents having a beneficial effect on the appearance of the skin, such as, for example, additional active agents intended to combat the signs of skin aging, or additional active agents intended to combat oily skin.

### Additional Active Agents

The composition according to the invention may also contain other active agents, and in particular at least one compound chosen from: moisturizing agents; depigmenting agents; anti-aging/anti-wrinkle agents (anti-glycation agents; NO-synthase inhibitors; agents for stimulating the synthesis of dermal or epidermal macromolecules and/or preventing degradation thereof; agents for stimulating fibroblast and/or keratinocyte proliferation or stimulating keratinocyte differentiation; muscle relaxants); agents having a restructuring effect on the cutaneous barrier function; agents for promoting maturation of the cornified envelope; agents for promoting the microcirculation of the skin, agents for stimulating the cellular energy metabolism of cells; tensioning agents; antioxidants; antipollution agents and/or free-radical scavengers, desquamating agents, sunscreens, and mixtures thereof.

The term “moisturizing agent” is intended to mean;
either a compound which acts on the barrier function, for the purpose of maintaining the moisturization of the stratum corneum, or an occlusive compound. Mention may be made of ceramides, sphingoid-based compounds, lecithins, glycosphingolipids, phospholipids, cholesterol and its derivatives, phytosterols (stigmasterol, β-sitosterol, campesterol), essential fatty acids, 1,2-diacylglycerol, 4-chromanone, pentacyclic triterpenes such as ursolic acid, petroleum jelly and lanolin;
or a compound which directly increases the water content of the stratum corneum, such as trehalose and derivatives thereof, hyaluronic acid and derivatives thereof, glycerol, pentanediol, sodium pidolate, serine, xylitol, sodium lactate, polyglyceryl acrylate, ectoin and derivatives thereof, chitosan, oligosaccharides and polysaccharides, cyclic carbonates, N-lauroylpyrrolidonecarboxylic acid, and N-α-benzoyl-L-arginine;
or a compound which activates the sebaceous glands, such as DHEA, 7-oxide and/or 17-alkyl derivatives thereof, sapogenins, and vitamin D and derivatives thereof.

The “depigmenting agents” that can be incorporated into the composition according to the present invention comprise, for example, the following compounds: kojic acid; ellagic acid; arbutin and derivatives thereof, such as those described in applications EP-895 779 and EP-524 109; hydroquinone; aminophenol derivatives such as those described in applications WO 99/10318 and Wo 99/32077, and in particular N-cholesteryloxycarbonyl-para-aminophenol and N-ethyloxycarbonylpara-aminophenol; iminophenol derivatives, in particular those described in application WO 99/22707; L-2-oxothiazolidine-4-carboxylic acid or procysteine, and also salts and esters thereof; ascorbic acid and derivatives thereof, in particular ascorbyl glucoside; and extracts of plants, in particular extracts of licorice, of mulberry or of skullcap, without this list being limiting.

The term “anti-glycation agent” is intended to mean a compound which prevents and/or reduces the glycation of skin proteins, in particular dermal proteins such as collagen.

Examples of anti-glycation agents are extracts of plants of the family Ericaceae, such as an extract of blueberry (Vaccinium angustifolium); ergothioneine and derivatives thereof; hydroxystilbenes and derivatives thereof, such as resveratrol and 3,3′,5,5′-tetrahydroxystilbene.

Examples of “NO-synthase inhibitors” that are suitable for use in the present invention comprise in particular an extract of a plant of the species Vitis vinifera, which is in particular sold by the company Euromed under the name Leucocyanidines de raisins extra, or else by the company Indena under the name Leucoselect®, or finally by the company Hansen under the name Extrait de marc de raisin; an extract of a plant of the species Olea europaea, which is preferably obtained from olive tree leaves and is in particular sold by the company Vinyals in the form of a dry extract, or by the company Biologia & Technologia under the trade name Eurol BT; and an extract of a plant of the species Gingko biloba, which is preferably a dry aqueous extract of this plant sold by the company Beaufour under the trade name Gingko biloba extract standard.

Among the “agents for stimulating dermal macromolecules or preventing degradation thereof”, mention may be made of those which act:
either on collagen synthesis, such as extracts of Centella asiatica; asiaticosides and derivatives; ascorbic acid or vitamin C and derivatives thereof; synthetic peptides such as iamin, the biopeptide CL or palmitoyl oligopeptide sold by the company Sederma; peptides extracted from plants, such as the soybean hydrolysate sold by the company Coletica under the trade name Phytokine®; and plant hormones such as auxins and lignans;
or on elastin synthesis, such as the extract of Saccharomyces cerivisiae sold by the company LSN under the trade name Cytovitin®; and the extract of the alga Macrocystis pyrifera sold by the company Secma under the trade name Kelpadelie®;
or on glycosaminoglycan synthesis, such as the product of fermentation of milk by Lactobacillus vulgaris, sold by the company Brooks under the trade name Biomin Yogourth®; the extract of the brown alga Padina pavonica sold by the company Alban Müller under the trade name HSP3®; and the extract of Saccharomyces cerevisiae available in particular from the company Silab under the trade name Firmalift® or from the company LSN under the trade name Cytovitin®;
or on fibronectin synthesis, such as the extract of the zooplankton Salina sold by the company Seporga under the trade name GP4G®; the yeast extract available in particular from the company Alban Müller under the trade name Drieline®; and the palmitoyl pentapeptide sold by the company Sederma under the tradename Matrixil®;
or on the inhibition of metalloproteinases (MMPs), such as more particularly MMP 1, 2, 3 or 9. Mention may be made of: retinoids and derivatives, oligopeptides and lipopeptides, lipoamino acids, the malt extract sold by the company Coletica under the trade name Collalift®; extracts of blueberry or of rosemary; lycopene; isoflavones, derivatives thereof or plant extracts containing them, in particular extracts of soybean, sold, for example, by the company Ichimaru Pharcos under the trade name Flavosterone SB®) of red clover, of flax, of kakkon or of sage;
or on the inhibition of serine proteases such as leucocyte elastase or cathepsin G. Mention may be made of: the peptide extract of legume seeds (Pisum sativum) sold by the company LSN under the trade name Parelastyl®; heparinoids; and pseudodipeptides such as {2-[acetyl(3-trifluoromethylphenyl)amino]-3-methylbutyrylamino}acetic acid.

Among the “active agents for stimulating epidermal macromolecules”, such as fillagrin and keratins, mention may in particular be made of the extract of lupin sold by the company Silab under the trade name Structurine®; the extract of Fagus sylvalica beech buds sold by the company Gattefossé under the trade name Gatuline®; and the extract of the zooplankton Salina sold by the company Seporga under the trade name GP4G®.

The “agents for stimulating fibroblast proliferation” that can be used in the composition according to the invention may, for example, be chosen from plant proteins or polypeptides, extracts, in particular of soybean (for example, an extract of soybean sold by the company LSN under the name Eleseryl SH-VEG 8® or sold by the company Silab under the trade name Raffermine®); and plant hormones such as giberrellins and cytokinins.

The “agents for stimulating keratinocyte proliferation” that can be used in the composition according to the invention comprise in particular retinoids such as retinol and esters thereof, including retinyl palmitate; phloroglucinol; extracts of walnut cakes sold by the company Gattefossé; and extracts of Solanum tuberosum sold by the company Sederma.

The “agents for stimulating keratinocyte differentiation” comprise, for example, minerals such as calcium; the extract of lupin sold by the company Silab under the trade name Photopréventine®; the sodium betasitosteryl sulfate sold by the company Seporga under the trade name Phytocohésine®; and the extract of corn sold by the company Solabia under the trade name Phytovityl®; and lignans such as secoisolariciresinol.

The composition according to the invention may comprise “dermodecontracting agents”, among which mention may in particular be made of alverine and salts thereof, in particular alverine citrate, sapogenins such as diosgenin and the natural extracts containing same (such as extracts of wild yam), certain carbonylated secondary and tertiary amines, organic or inorganic metal salts, in particular manganese gluconate, adenosine, and also the hexapeptide argireline R sold by the company Lipotec. Mention may also be made of certain fragrancing compositions with a dermodecontracting effect.

As “agents having a restructuring effect on the cutaneous barrier” mention may be made of an extract of Thermus thermophilus, such as Vénucéane® from Sederma, an extract of rhizome of wild yam (Dioscorea villosa) such as Actigen Y® from Active Organics, plankton extracts, such as omega plankton® from Secma, yeast extracts, such as Relipidium® from Coletica, a chestnut extract, such as Recoverine® from Silab, a cedar bud extract such as Gatuline Zen® from Gattefosse, Phytosphingosine SLC from Degussa, Aquaxyl® from Seppic and Lipidessence® from Coletica.

Mention may also be made, in particular, of ceramides and derivatives, sphingoid-based compounds, glycosphingolipids, phospholipids, cholesterol and derivatives thereof, phytosterols, essential fatty acids, diacylglycerol, 4-chromanone and chromon derivatives, petroleum jelly, lanolin, shea butters, cocoa butter, etc.

As preferred additional agents for promoting the cutaneous barrier function, mention will be made of an extract of Thermus thermophilus, an extract of rhizome of wild yam (Dioscorea villosa), a yeast extract, a chestnut extract, a cedar bud extract, and mixtures thereof.

By way of “agents for promoting maturation of the cornified envelope”, use may be made, in the compositions of the invention, of agents that participate in the maturation of the cornified envelope, which becomes impaired with age and induces a decrease in transglutaminase activity. Mention may, for example, be made of urea and derivatives thereof, and in particular Hydrovance® from National Starch and the other active agents mentioned in L'Oréal application FR2877220.

By way of “agents for stimulating the microcirculation of the skin”, it is possible to combine, in the compositions of the invention, agents that act on the microcirculation of the skin in order to prevent dulling of the complexion and/or the formation of pockets, for instance an extract of black tea such as Kombuchka from Sederma, Pycnogenol, manganese gluconate (Givobio GMn from Seppic), Visnadine from Indena, an extract of lupin (Eclaline from Silab), Epaline 100 from Laboratoires Carilène, an extract of bitter orange flower (Remoduline from Silab), vitamin P and derivatives thereof, such as Permethol from Sochibios and other extracts (of ruscus, of horsechestnut, of ivy, of ginseng, of melilot, etc.) and also caffeine, escin, hesperitine laurate, dextran sulfate, nicotinate and derivatives, lysine and derivatives (such as Asparlyne from Solabia), etc.

As preferred additional agents for promoting the microcirculation of the skin, mention will be made of Kombuchka, manganese gluconate, and vitamin P and derivatives thereof.

For “agents for stimulating the energy metabolism of cells”, it is possible to also combine active agents for stimulating energy metabolism which becomes slowed down during aging, among which mention may be made of biotin, an extract of Saccharomyces cerevisiae, such as Phosphovital® from Sederma, Physiogenyl® from Solabia, a mixture of zinc, copper and magnesium gluconate such as Sepitonic M3® from Seppic.

The “tensioning agent” is intended to mean a compound capable of exerting a tensile force on the skin, the effect of which is to temporarily fade out the irregularities of the surface of the skin, such as wrinkles and fine lines.

By way of “antioxidants”, mention may in particular be made of tocopherol and esters thereof, in particular tocopheryl acetate; BHT and BHA.

Mention may also be made of polyphenols, tannic acid, epigallocatechins and natural extracts containing them, anthocyans, extracts of rosemary, extracts of olive tree leaves, green tea, resveratrol and derivatives thereof, Pycnogenol, ergothioneine, N-acetylcysteine, biotin, chelating agents, idebenone, plant extracts such as Pronalen Bioprotect™ from the company Provital, free-radical scavengers such as vitamin E, coenzyme Q10, bioflavonoids, SODs, phytantriol, lignans, melatonin, pidolates and glutathione.

The expression “antipollution agent” is intended to mean any compound capable of trapping ozone, monocyclic or polycyclic aromatic compounds such as benzopyrene and/or heavy metals such as cobalt, mercury, cadmium and/or nickel. The term “free-radical scavenger” is intended to mean any compound capable of trapping free radicals.

As “ozone-trapping agents” that can be used in the composition according to the invention, mention may in particular be made of vitamin C and derivatives thereof, including ascorbyl glucoside; phenols and polyphenols, in particular tannins, ellagic acid and tannic acid; epigallocatechin and natural extracts containing it; extracts of olive tree leaf; extracts of tea, in particular of green tea; anthocyans; extracts of rosemary; phenol acids, in particular chlorogenic acid; stilbenes, in particular resveratrol; sulfur-containing amino acid derivatives, in particular S-carboxymethylcysteine; ergothioneine; N-acetylcysteine; chelating agents such as N,N′-bis(3,4,5-trimethoxybenzyl)ethylenediamine or one of its salts, metal complexes or esters; carotenoids such as crocetin; and various starting materials such as the mixture of arginine, histidine ribonucleate, mannitol, adenosine triphosphate, pyridoxine, phenylalanine, tyrosine and hydrolyzed RNA sold by Laboratoires Sérobiologiques under the trade name CPP LS 2633-12F®, the water-soluble fraction of corn sold by the company Solabia under the trade name Phytovityl®, the mixture of extract of fumitory and of extract of lemon sold under the name Unicotrozon C-49® by the company Induchem, and the mixture of extracts of ginseng, of apple, of peach, of wheat and of barley sold by the company Provital under the trade name Pronalen Bioprotect®.

As “agents for trapping monocyclic or polycyclic aromatic compounds” that can be used in the composition according to the invention, mention may in particular be made of tannins such as ellagic acid; indole derivatives, in particular indole-3-carbinol; extracts of tea, in particular of green tea, extracts of water hyacinth or Eichornia crassipes; and the water-soluble fraction of corn sold by the company Solabia under the trade name Phytovityl®.

Finally, as “agents for trapping heavy metals” that can be used in the composition according to the invention, mention may in particular be made of chelating agents such as EDTA, the pentasodium salt of ethylenendiaminetetramethylenephosphonic acid, and N,N′-bis(3,4,5-trimethoxybenzyl)ethylenediamine or one of its salts, metal complexes or esters; phytic acid; chitosan derivatives; extracts of tea, in particular of green tea; tannins such as ellagic acid; sulfur-containing amino acids such as cysteine; extracts of water hyacinth (Eichornia crassipes); and the water-soluble fraction of corn sold by the company Solabia under the trade name Phytovityl®.

The “free-radical scavengers” that can be used in the composition according to the invention comprise, in addition to certain antipollution agents mentioned above, vitamin E and derivatives thereof, such as tocopheryl acetate; bioflavonoids; coenzyme Q10 or ubiquinone; certain enzymes such as catalase, superoxide dismutase, lactoperoxidase, glutathione peroxidase and quinone reductases; glutathione; benzylidenecamphor; benzylcyclanones; substituted naphthalenones; pidolates; phytantriol; gamma-oryzanol; lignans; and melatonin.

The term “desquamating agent” is intended to mean any compound capable of acting:
either directly on desquamation by promoting exfoliation, such as β-hydroxy acids, in particular salicylic acid and its derivatives (including 5-n-octanoylsalicylic acid); α-hydroxy acids, such as glycolic acid, citric acid, lactic acid, tartaric acid, malic acid or mandelic acid; urea; gentisic acid; oligofucoses; cinnamic acid; extract of Saphora japonica; resveratrol;
or on the enzymes involved in desquamation or the degradation of corneodesmosomes, such as glycosidases, stratum corneum chymotryptic enzyme (SCCE), or even other proteases (trypsin, chymotrypsin-like), Mention may be made of agents for chelating mineral salts: EDTA; N-acyl-N,N′,N′-ethylenediaminetriacetic acid; aminosulfonic compounds, and in particular N-(2-hydroxyethyl)piperazine-N′-2-ethanesulfonic acid (HEPES); derivatives of 2-oxothiazolidine-4-carboxylic acid (procysteine); derivatives of alpha-amino acids of glycine type (as described in EP-0 852 949, and also the sodium methyl glycine diacetate sold by BASF under the trade name Trilon M>; honey; and sugar derivatives such as O-octanoyl-6-D-maltose and N-acetylglucosamine.

The invention also relates to a cosmetic treatment process for the skin for improving and/or reinforcing the barrier function of the skin, comprising at least one step consisting in applying at least one composition as defined above to the skin.

According to a first embodiment, the process according to the invention comprises at least one step consisting in applying at least one composition as defined above to the skin of individuals who have skin exhibiting at least one of the signs of skin aging recalled above.

According to another embodiment, it comprises at least one step consisting in applying at least one composition as defined above to the skin of individuals who have skin or an area of skin that has been stressed, in particular shaved skin of the face or the body.

### Cosmetic Assembly

According to another aspect, the invention also relates to a cosmetic assembly comprising:
i) a container delimiting at least one compartment, said container being closed by means of a closing member; and
ii) a composition as described above and placed inside said compartment.

The container may be in any appropriate form. It may in particular be in the form of a bottle, a tube, a jar, a case, a box, a sachet or a carton.

The closing member may be in the form of a removable stopper, a lid, a cap, a tear-off strip or a capsule, in particular of the type comprising a body attached to the container and a cover cap articulated on the body. It may also be in the form of a member for selectively closing the container, in particular a pump, a valve or a flap valve.

The container may be combined with an applicator. The applicator may be in the form of a brush, as described, for example, in patent FR 2 722 380. The applicator may be in the form of a block of foam or of elastomer, a felt or a spatula. The applicator may be free (tuft or sponge) or securely fastened to a rod borne by the closing member, as described, for example, in U.S. Pat. No. 5,492,426. The applicator may be securely fastened to the container, as described, for example, in patent FR 2 761 959.

The product may be contained directly in the container, or indirectly. By way of example, the product may be arranged on an impregnated support, in particular in the form of a wipe or of a pad, and arranged (individually or in plurality) in a box or in a sachet. Such a support incorporating the product is described, for example, in application WO 01/03538.

The closing member may be coupled to the container by screwing. Alternatively, the coupling between the closing member and the container is done other than by screwing, in particular via a bayonet mechanism, by click-fastening, gripping, welding, bonding or by magnetic attraction. The term “click-fastening” is intended to mean in particular any system involving the crossing of a bead or cord of material by elastic deformation of a portion, in particular of the closing member, followed by return to the elastically unconstrained position of said portion after crossing of the bead or cord.

The container may be at least partially made of thermoplastic material. By way of examples of thermoplastic material, mention may be made of polypropylene or polyethylene.

Alternatively, the container is made of nonthermoplastic material, in particular glass or metal (or alloy).

The container may have rigid walls or deformable walls, in particular in the form of a tube or a tubular bottle.

The container may comprise means for initiating or facilitating the distribution of the composition. By way of example, the container may have deformable walls so as to allow the composition to exit in response to a positive pressure inside the container, this positive pressure being caused by elastic (or nonelastic) squeezing of the walls of the container.

The container may consist of a carton with a base delimiting at least one housing containing the composition, and a lid, in particular articulated on the base, and capable of at least partially covering said base. Such a carton is described, for example, in application WO 03/018423 or in patent FR 2 791 042.

The container may be equipped with a drainer arranged in the region of the aperture of the container. Such a drainer makes it possible to wipe the applicator and possibly the rod to which it may be securely fastened. Such a drainer is described, for example, in patent FR 2 792 618.

The content of the patents or patent applications mentioned above is incorporated into the present application by way of reference.

According to a specific embodiment, the invention relates to a cosmetic assembly comprising:
a composition A containing at least one C-glycoside derivative,
a composition B, packaged separately from composition A, comprising at least one hyaluronic acid or a derivative thereof.

Finally, the invention relates to a cosmetic treatment process comprising at least one step of applying composition A to the skin and/or its appendages and at least one step of applying composition B to the skin and/or its appendages.

According to a preferred embodiment, composition A is firstly applied and then composition B is applied.

Without it binding them to any theory, the inventors see an advantage in this order of application, originating from the fact that the C-glycoside derivative prepares the skin and/or its appendages to respond to a greater extent to the hyaluronic acid or to derivatives thereof.

In this case, compositions A and B can be packaged separately inside two compartments, either formed by two separate containers, or inside a single device.

The term “single device” is intended to mean a device by means of which the two compartments are securely fastened to one another. Such a device may be obtained by a process of molding the two compartments in a single piece, in particular made of a thermoplastic material. It may also result from any form of assembly, in particular by bonding, welding, or another click-fastening.

According to a first embodiment, the two containers are independent of one another. Such containers may be in various forms. They may in particular be tubes, bottles or flasks.

One and/or the other of the containers may be surmounted by a manually actuated pump surmounted by a pushbutton for actuating the pump and the distribution of the composition via at least one distribution orifice.

Alternatively, one and/or the other of the containers is (are) pressurized, especially by means of a propellant, in particular a propellant gas. In this case, the container(s) is (are) equipped with a valve surmounted by a pushbutton equipped with a nozzle or with any other diffusion means for dispensing the product.

The propellant may be in a mixture with the composition to be dispensed or separated, in particular via a piston capable of sliding inside the container, or via the flexible walls of a pocket inside of which the composition is placed.

The containers may consist of various materials: plastic, glass or metal.

Again alternatively, the two compartments are formed from two concentric compartments formed inside a tube, and are surmounted by a non-return air pump equipped with a pushbutton with one or two dispensing orifices. A piston is provided inside the tube, said piston travelling back in the direction of the pump as the compositions are removed from inside the containers. Such dispensing methods are used in particular for dispensing toothpastes.

The examples which appear hereinafter illustrate the present invention.

### EXAMPLES

The C-glycoside derivative used is the C-β-D-xylopyranoside-2-hydroxypropane sold under the name Mexoryl® by Chimex. It is in the form of a solution at 30% by weight with respect to active material in a 60/40 water/1,2-propanediol mixture. The hyaluronic acid used is Cristalhyal® AL sold by the company Solliance (MW: 1.1×10⁶ Da).

### Example 1

### Test Carried Out

### Assaying of Keratinocyte Transglutaminase (TGK) Activity

The in vitro effect of 2 active agents was studied (C-β-D-xylopyranoside-2-hydroxypropane at 30% by weight with respect to active material in a 60/40 water/1,2-propanediol mixture and hyaluronic acid) on keratinocyte differentiation. For this, normal human epidermal keratinocytes (NHEKs), pretreated with mitomycin so as to prevent proliferation thereof, in a deficient medium made up of 40% of depleted SFM medium and 60% of Earle's balanced salt solution (EBSS) buffer were placed in culture. The metabolism of these cells having been thus slowed down, their ability to differentiate is then quantified by assaying, using a standard radiochemical assay, the keratinocyte transglutaminase activity (TGk) of the NHEKs pretreated with the active agents.

Calcium chloride (CaCl₂), a molecule known to stimulate keratinocyte differentiation, was used as reference molecule for this assay.

### Procedure

Normal human keratinocytes were cultured at 37° C. and 5% CO₂ for 24 h in complete SFM medium. The cell divisions were then blocked by treatment with mitomycin C (10 μg/ml) for 2 hours at 37° C. and 5% CO₂. The cells were then incubated for 96 hours in deficient or nondeficient (control) medium containing or not containing various concentrations of the test or reference products. In parallel, 1,2-propanediol, the solvent for C-β-D-xylopyranoside-2-hydroxypropane, was tested. The treatments were carried out in triplicate (n=3).

At the end of the incubation, the TGk membrane enzyme was extracted in the presence of Triton X-100 and the TGk activity was determined by measuring the covalent addition of tritiated putrescine, at a final concentration of 2 μCi/ml, to caseine (acceptor protein, 2 mg/ml final concentration).

The caseine was precipitated by addition of trichloroacetic acid (TCA) and the precipitates were recovered on filters and a collector and then the dry filters were counted by liquid scintillation.

### Results

TGk activity—medium with mitomycin

| | | % | |
|---|---|---|---|
| | | Activity | Significant |
| | | relative | variation |
| | | to the | relative to |
| TREATMENT | CONCENTRATION | control | the control |
| Depleted control | — | 100 | — |
| medium | | | |
| (40% SFM [+Ep + EGF] + | | | |
| 60% EBSS [+Ca + Mg]) | | | |
| Normal medium control | — | 209 | +109% |
| (100% SFM [+Ep + EGF]) | | | |
| CaCl₂ | 1.5 mM | 557 | +457% |
| Hyaluronic acid⁽¹⁾ | 0.02% | 88 | NS |
| C-β-D-xylopyranoside-2- | 0.06% | 128 | NS |
| hydroxypropane⁽²⁾ | | | |
| 1,2-propanediol | 0.16% | 87 | NS |
| Hyaluronic acid⁽¹⁾ and | 0.02% and | 222 | +122% |
| C-β-D-xylopyranoside-2- | 0.06% | | |
| hydroxypropane⁽²⁾ | | | |

(NS = not significant)
⁽¹⁾Cristalhyal AL sold by the company Solliance (MW: 1.1 × 10⁶ Da)
⁽²⁾Mexoryl SBB ® manufactured by Chimex

In this assay, calcium chloride at 1.5 mM, used as reference molecule, clearly increased the TGk activity of the keratinocytes. This result was in agreement with the expected effects and made it possible to validate the assay.

The C-β-D-xylopyranoside-2-hydroxypropane tested at 0.06% has no significant effect on the TGk activity.

Its solvents 1,2-propanediol, tested at 0.8%, did not modify the activity of this enzyme either.

Hyaluronic acid tested alone at 0.02% also has no effect on the keratinocyte transglutaminase activity.

The combination of C-β-D-xylopyranoside-2-hydroxypropane and hyaluronic acid, tested respectively at 0.06% and 0.02%, showed a synergistic effect, insofar as the products tested alone and at this concentration do not modify the TGk activity, whereas the combination significantly stimulated the activity. The same effects were observed when the activity was related to the amount of proteins.

### Conclusion

The C-β-D-xylopyranoside-2-hydroxypropane/hyaluronic acid mixture increases the keratinocyte transglutaminase activity in normal human epidermal keratinocytes cultured in deficient medium.

The effects of C-β-D-Xylopyranoside-2-hydroxypropane are potentiated by hyaluronic acid and are similar to those observed if the cells were cultured in normal medium.

The results obtained for this combination of active agents reflect an increase in epidermal differentiation.

### Example 2

### Formulation Examples

The contents are indicated as percentage by mass.

Compositions A and B are O/W emulsions gelled with a carbomer and/or ammonium polyacryloyldimethyl taurate, and are prepared according to the usual techniques. Composition B is very highly siliconized. Composition C is a highly siliconized W/O emulsion prepared according to the usual techniques. Composition D is a transparent gel which has a high concentration of hyaluronic acid and is gelled with ammonium polyacryloyldimethyl taurate,

| | A | B | C | D |
|---|---|---|---|---|
| Magnesium sulfate | | | 0.70 | |
| Sodium hydroxide | 0.08 | | | |
| Triethanolamine | | 1.23 | 0.01 | 0.02 |
| Hydroxyethylpiperazine- | 1.00 | | | 1.00 |
| ethanesulfonic acid | | | | |
| C-β-D-xylopyranoside- | 1.00 | 1.00 | 1.00 | 1.00 |
| 2-hydroxypropane⁽¹⁾ | | | | |
| Algal extracts | 1.00 | 0.20 | 0.20 | 0.20 |
| Disteardimonium hectorite | | | 0.20 | |
| Silica | | 2.00 | 3.00 | |
| Acrylate copolymer | | | 0.30 | |
| Tristearine (and) acetylated | | | 1.00 | |
| glycol stearate | | | | |
| Caprylic/capric triglyceride | | 3.00 | | |
| Phenoxyethanol | 0.60 | | 0.70 | |
| Methylparaben | 0.20 | | | |
| Chlorphenesine | 0.30 | | | |
| Ethylhexyl methoxycinnamate | 1.00 | | 1.00 | |
| Phenylbenzimidazolesulfonic | | 1.80 | | |
| acid | | | | |
| Ethylhexyl methoxycinnamate | | 7.50 | | |
| Titanium dioxide (and) mica | 1.00 | 1.00 | 1.00 | |
| Hydroxyethylcellulose | | | | 0.12 |
| Sodium hyaluronate⁽²⁾ | 0.02 | 0.02 | 0.02 | 0.50 |
| Carbomer | 0.15 | | | |
| Ammonium polyacryloyldimethyl | 1.00 | 1.60 | | 0.25 |
| taurate | | | | |
| Poly(methyl methacrylate) | | 2.00 | | |
| HDI/Trimethylolhexyllactone | | 1.00 | | |
| crosspolymer | | | | |
| Cyclohexasiloxane | | 3.00 | 22.00 | |
| Cetyl PEG/PPG-10/1 dimethicone | | | 3.00 | |
| Dimethicone (and) | 10.00 | | | |
| dimethicone/vinyl dimethicone | | | | |
| crosspolymer | | | | |
| Bis-PEG-18 methyl ether | | | | 3.00 |
| dimethyl silane | | | | |
| Stearyl dimethicone | 2.00 | | | |
| Cyclopentasiloxane (and) | | 2.00 | | |
| dimethiconol | | | | |
| Dimethicone (and) dimethiconol | | 3.00 | | |
| Cyclopentasiloxane (and) | 25.00 | | | |
| dimethicone crosspolymer | | | | |
| Divinyl | | 0.50 | | |
| dimethicone/dimethicone | | | | |
| crosspolymer (and) C12-13 | | | | |
| pareth-23 (and) C12-13 | | | | |
| pareth-3 | | | | |
| Dimethicone (and) ceteth-10 | 3.50 | | | |
| (and) Laureth-4 | | | | |
| Denatured alcohol | | 5.00 | 5.00 | 7.00 |
| Water | 38.15 | 55.15 | 45.87 | 79.41 |
| Glycerol | 7.00 | 7.00 | 10.00 | 7.00 |
| Propylene glycol | 6.00 | | 4.00 | |
| Caprylyl glycol | | | | 0.50 |
| Polysorbate 80 | 1.00 | | | |
| Methyl glucose sesquistearate | | 2.00 | | |
| PEG-20 | | | | |
| Polyglyceryl-4 isostearate | | | 1.00 | |

⁽¹⁾Mexoryl SBB ® manufactured by Chimex
⁽²⁾Cristalhyal AL ® sold by Solliance

1-22. (canceled)

23. A topical cosmetic and/or dermatological composition comprising, in a physiologically acceptable medium, at least one hyaluronic acid or a derivative thereof and at least one C-glycoside derivative.

24. The composition as claimed in claim 23, in which the hyaluronic acid or a derivative thereof has a molecular weight of between 50 000 and 5 000 000 Da.

25. The composition as claimed in claim 23 in which the hyaluronic acid or a derivative thereof has a molecular weight of between 100 000 and 5 000 000 Da.

26. The composition as claimed in claim 23 in which the hyaluronic acid or a derivative thereof has a molecular weight of between 400 000 and 5 000 000 Da.

27. The composition as claimed in claim 23, in which the hyaluronic acid or a derivative thereof is present at a content of between 0.001% and 20% by weight, of active material of hyaluronic acid or a derivative thereof, relative to the total weight of the composition.

28. The composition as claimed in claim 23, in which the hyaluronic acid or a derivative thereof is present at a content of between 0.01% and 10% by weight, of active material of hyaluronic acid or a derivative thereof, relative to the total weight of the composition.

29. The composition as claimed in claim 23, in which the hyaluronic acid or a derivative thereof is present at a content of between 0.01% and 5% by weight, of active material of hyaluronic acid or a derivative thereof, relative to the total weight of the composition.

30. The composition as claimed in claim 23, in which the C-glycoside derivative corresponds to general formula (I) below: 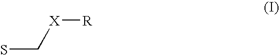
in which:
R represents:
a C₁ to C₂₀, saturated or C₂ to C₂₀, unsaturated, linear alkyl radical, or a C₃ to C₂₀, saturated or unsaturated, branched or cyclic alkyl radical;
a C₁ to C₂₀, saturated or C₂ to C₂₀, unsaturated, linear, or C₃ to C₂₀, saturated or unsaturated, branched or cyclic, hydrofluoro- or perfluoroalkyl radical;
it being possible for the hydrocarbon-based chain constituting said radicals to be, where appropriate, interrupted with 1, 2, 3 or more heteroatoms chosen from:
an oxygen,
a sulfur,
a nitrogen, and
a silicon,
and it being possible for it to be optionally substituted with at least one radical chosen from:
—OR₄,
—SR₄,
—NR₄R₅,
—COOR₄,
—CONHR₄,
—CN,
a halogen atom,
a C₁ to C₆ hydrofluoro- or perfluoroalkyl radical and/or
a C₃ to C₈ cycloalkyl radical,
with R₄ and R₅ possibly representing, independently of one another, a hydrogen atom, or a C₁ to C₃₀, saturated or C₂ to C₃₀, unsaturated, linear, or C₃ to C₃₀, saturated or unsaturated, branched or cyclic, alkyl, perfluoroalkyl or hydrofluoroalkyl radical; or a C₆ to C₁₀ aryl radical,
X represents a radical chosen from the groups: 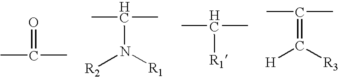
with R₁, R₇ and R₃ representing, independently of one another, a hydrogen atom, or a radical R, with R as defined above, and R′¹ representing a hydrogen atom, an —OH group or a radical R as defined above, R₁ also possibly denoting a C₆ to C₁₀ aryl radical;
S represents a monosaccharide or a polysaccharide comprising up to 20 sugar units, in pyranose and/or furanose form of the L and/or D series, it being possible for said monosaccharide or polysaccharide to be substituted with a hydroxyl group which is necessarily free, and optionally one or more optionally protected amine function(s), and
the S—CH₂—X bond represents a bond of C-anomeric nature which may be a or P,
and also the cosmetically acceptable salts thereof, the solvates thereof, such as hydrates, and the isomers thereof.

31. The composition as claimed in claim 30, in which S represents a monosaccharide chosen from D-glucose, D-xylose, L-fucose, D-galactose and D-maltose.

32. The composition as claimed in claim 31, in which X represents a group chosen from —CO—, —CH(OH)— and —CH(NH₂)—.

33. The composition as claimed in claim 32, in which R denotes a C₁-C₄, in particular C₁-C₃, linear radical optionally substituted with —OH, —COOH or —COOR″₂, R″2 being a C₁-C₄ saturated alkyl radical.

34. The composition as claimed in claim 23, in which the C-glycoside derivative is chosen from:
C-β-D-xylopyranoside-n-propan-2-one,
C-α-D-xylopyranoside-n-propan-2-one,
C-β-D-xylopyranoside-2-hydroxypropane,
C-α-D-xylopyranoside-2-hydroxypropane,
1-(C-β-D-fucopyranoside)propan-2-one,
1-(C-α-D-fucopyranoside)propan-2-one,
1-(C-β-L-fucopyranoside)propan-2-one,
1-(C-α-L-fucopyranoside)propan-2-one,
1-(C-β-D-fucopyranoside)-2-hydroxypropane,
1-(C-α-D-fucopyranoside)-2-hydroxypropane,
1-(C-β-L-fucopyranoside)-2-hydroxypropane,
1-(C-α-L-fucopyranoside)-2-hydroxypropane,
1-(C-β-D-glucopyranosyl)-2-hydroxypropane,
1-(C-α-D-glucopyranosyl)-2-hydroxypropane,
1-(C-β-D-galactopyranosyl)-2-hydroxypropane,
1-(C-α-D-galactopyranosyl)-2-hydroxypropane,
1-(C-β-D-fucofuranosylpropan-2-one,
1-(C-α-D-fucofuranosyl)propan-2-one,
1-(C-β-L-fucofuranosyl)propan-2-one,
1-(C-α-L-fucofuranosyl)propan-2-one,
C-β-D-maltopyranoside-n-propan-2-one,
C-α-D-maltopyranoside-n-propan-2-one,
C-β-D-maltopyranoside-2-hydroxypropane,
C-α-D-maltopyranoside-2-hydroxypropane, isomers thereof and mixtures thereof.

35. The composition as claimed in claim 23, in which the C-glycoside derivative is chosen from C-β-D-xylopyranoside-2-hydroxypropane and C-α-D-xylopyranoside-2-hydroxypropane.

36. The composition as claimed in claim 23, wherein the C-glycoside derivative is present at a content ranging from 0.0001% to 25% by weight of active material of C-glycoside derivative, relative to the total weight of the composition.

37. The composition as claimed in claim 23, wherein the C-glycoside derivative is present at a content ranging from 0.001% to 10% by weight of active material of C-glycoside derivative, relative to the total weight of the composition.

38. The composition as claimed in claim 23, wherein the C-glycoside derivative is present at a content ranging from 0.05% to 5% by weight of active material of C-glycoside derivative, relative to the total weight of the composition.

39. The composition as claimed in claim 23, in which the ratio by weight of the hyaluronic acid or a derivative thereof to the C-glycoside derivative ranges between 0.001 and 1000.

40. The composition as claimed in claim 23, in which the ratio by weight of the hyaluronic acid or a derivative thereof to the C-glycoside derivative ranges between 1 and 10.

41. The composition as claimed in claim 23, in which the ratio by weight of the hyaluronic acid or a derivative thereof to the C-glycoside derivative ranges between 2 and 4.

42. A cosmetic assembly comprising:
a composition A comprising at least one C-glycoside derivative,
a composition B comprising at least one hyaluronic acid derivative or a derivative thereof.

43. Method for using a cosmetic composition as defined in claim 23 for improving and/or reinforcing the barrier function of the skin.

44. Method for using a cosmetic composition as defined in claim 23 for improving and/or reinforcing the protection of the skin against outside attacks.

45. Method for using a cosmetic composition as defined in claim 23 for improving the hydration of the skin and/or its appendages.

46. Method for using a composition as defined in claim 23 for preventing and/or treating the roughness or the microrelief of the skin and/or its appendages and/or for improving the radiance of the complexion and/or for improving the suppleness of the skin and/or its appendages.

47. Method for using a cosmetic composition as defined in claim 23 for smoothing out the signs of skin aging.

48. Method as claimed in the claim 47, wherein the signs of skin aging are chosen from impairment of the viscoelastic or biomechanical properties of the skin, impairment of tissue cohesion, thinning of the skin, the appearance of wrinkles and/or fine lines, the appearance of browning and/or of yellowing of the skin and the appearance of age or senescence or lentigo spots.

49. A cosmetic treatment process for improving and/or reinforcing the barrier function and/or the hydration and/or the resistance to outside attacks of the skin and/or its appendages, and/or for combating the signs of skin aging, and wherein an effective amount of at least one C-glycoside derivative and of at least one hyaluronic acid or a derivative thereof is applied to the skin and/or its appendages.

50. A cosmetic treatment process comprising at least one step of applying composition A as defined in claim 42 to the skin and/or its appendages and at least one step of applying composition B as defined in claim 42 to the skin and/or its appendages.

51. Method of using at least one hyaluronic acid or a derivative thereof and of at least one C-glycoside derivative, for the preparation of a topical dermatological composition for improving and/or reinforcing the barrier function of the skin.

52. Method for using at least one hyaluronic acid or a derivative thereof and of at least one C-glycoside derivative, for the preparation of a topical dermatological composition for improving and/or reinforcing the barrier function of damaged skin.

53. Method for using at least one hyaluronic acid or a derivative thereof and of at least one C-glycoside derivative, for the preparation of a topical dermatological composition for improving and/or reinforcing the barrier function of damaged skin requiring tissue repair and/or regeneration.
